(19) United States
(12) Patent Application Publication
(10) Pub. No.: US 2024/0016377 A1
(43) Pub. Date: Jan. 18, 2024

(54) SYSTEMS AND METHODS FOR OPTICAL ILLUMINATION IN A SPECULUM TIP

(71) Applicant: OtoNexus Medical Technologies, Inc., Bellevue, WA (US)

(72) Inventors: Anthony J. Alleman, Bellevue, WA (US); Brent Christopher Mayer, Bellevue, WA (US); Nicholas E. Bratt, Bellevue, WA (US); David W. Rush, Bellevue, WA (US)

(73) Assignee: OtoNexus Medical Technologies, Inc., Bellevue, WA (US)

(21) Appl. No.: 18/476,263

(22) Filed: Sep. 27, 2023

Related U.S. Application Data
(63) Continuation of application No. PCT/US22/35003, filed on Jun. 24, 2022.
(60) Provisional application No. 63/214,938, filed on Jun. 25, 2021.

(51) Int. Cl. A61B 1/32; A61B 1/227; A61B 1/07; A61B 8/12; A61B 8/00; A61B 1/00

(52) U.S. Cl. CPC A61B 1/32; A61B 1/227; A61B 1/07; A61B 8/12; A61B 8/4416; A61B 1/00126

(57) ABSTRACT

A speculum operable to be disposed within an ear of a subject may include a housing. The housing may include a light conducting element. A transmitted optical illumination may be conducted by total internal reflection via the light conducting element. The housing may include a lumen. The housing may be configured to allow a reflected optical illumination to propagate through the lumen. The speculum may include one or more coupling portions which couple the transmitted optical illumination from a light source to the light conducting element. The one or more coupling portions may be shaped as a conic section.

### CROSS-REFERENCE

This application is a continuation of PCT Application No. PCT/US2022/35003, filed Jun. 24, 2022, which claims benefit of U.S. Provisional Patent Application No. 63/214,938 filed Jun. 25, 2021, which is entirely incorporated by reference.

### BACKGROUND

Acute otitis media (AOM) is an inflammatory process in the middle ear and is the most common clinical condition seen by pediatricians in children fifteen years and younger. AOM is generally associated with the presence of a middle ear effusion and is considered a middle ear inflammation. Complications of undiagnosed AOM can include hearing loss. Left untreated in children, recurrent AOM can also lead to delays in the development of speech and language skills.

The likelihood of obtaining an accurate diagnosis using existing non-invasive methods may be no better than 50%. Further, existing non-invasive methods may only be useful in identifying the presence of an effusion, and they often provide no information regarding the type of effusion. Because of the risks associated with undiagnosed AOM and the recognized unreliability of existing diagnostic tests, patients are often prescribed antibiotics, which may be ineffective in treating viral effusion. In addition to the increased cost burden of unnecessary antibiotic treatment, the patients are exposed to the side effects of antibiotics and the attendant and significant risk of developing antibiotic resistance.

### SUMMARY

Devices and methods described herein may improve upon existing non-invasive techniques by measuring ultrasound data reflected from a biological membrane coincident with a pneumatic excitation. The size of a diagnostic target may be small, and ultrasound is not visible to a human eye. An optical source and detection system may be provided. The optical source and detection system may facilitate alignment of the ultrasound beam. As the size of a biological lumen may be small, the optical source and detection system may be space efficient. The present disclosure provides improvements to delivering optical illumination to a target. The present disclosure provides improvements receiving optical illumination from a target in the presence of an obstruction.

In an aspect, the present disclosure provides a speculum operable to be disposed within an ear of a subject. The speculum may comprise a housing comprising a light conducting element, wherein a transmitted optical illumination is conducted by total internal reflection via the light conducting element, wherein the housing has a lumen therewithin, and wherein the housing is configured to allow a reflected optical illumination to propagate through the lumen; and one or more coupling portions which couple the transmitted optical illumination from a light source to the light conducting element, wherein the one or more coupling portions are shaped as a conic section.

In another aspect, the present disclosure provides an otoscope. The otoscope may comprise: a speculum and having a lumen therewithin and comprising a light conducting element, wherein a transmitted optical illumination is conducted by total internal reflection by the light conducting element, and wherein a reflected optical illumination is propagated through the lumen of the speculum; and one or more coupling portions which couple the transmitted optical illumination from a light source to the light conducting element, wherein the one or more coupling portions are shaped as a conic section.

In another aspect, the present disclosure provides a method of using an otoscope. The method may comprise: directing optical illumination of a light source to one or more coupling portions, wherein the one or more coupling portions are shaped as a conic section; collimating the optical illumination using the one or more coupling portions; directing the optical illumination from the one or more coupling portions to a light conducting element, wherein the optical illumination is conducted by total internal reflection by the light conducting element; and collecting reflected optical illumination from a target within a lumen of the housing, wherein the housing comprises a portion of a speculum of an otoscope.

In some embodiments, the method further comprises directing pneumatic excitation toward the target. In some embodiments, the method further comprises directing ultrasound and/or illumination toward the target. In some embodiments, the method further comprises measuring a response of the target to the pneumatic excitation in a reflected ultrasound signal. In some embodiments, the method further comprises determining a state or condition of a subject based on the reflected optical illumination and the response.

In another aspect, the disclosure provides a speculum operable to be disposed within an ear of a subject, the speculum comprising: a housing comprising a light conducting element, wherein a transmitted optical illumination is conducted by total internal reflection via the light conducting element, wherein the housing has a lumen therewithin, and wherein the housing is configured to allow a reflected optical illumination to propagate through the lumen; and one or more coupling portions which couple the transmitted optical illumination from a light source to the light conducting element, wherein the one or more coupling portions are shaped as a conic section. In some embodiments, the device further comprises an insert, wherein the insert is configured to be mechanically coupled to the housing. In some embodiments, the insert comprises a lens, an ultrasound transducer, one or more electrical leads electrically coupled to the ultrasound transducer, one or more wires electrically coupled to the one or more electrical leads and the ultrasound transducer, or any combination thereof. In some embodiments, the ultrasound transducer comprises a capacitive micromachined ultrasonic transducer. In some embodiments, the light conducting element comprises an ellipsoid shape. In some embodiments, the light conducting element is configured to be a parabolic mirror when light rays of the light source interact with the light conducting element. In some embodiments, the light conducting element comprises a launch point. In some embodiments, the light conducting element comprises one or more launch points. In some embodiments, the launch points comprise a geometry, wherein the geometry comprises: flat, circular, oval, trough, square, flat, or V-shaped. In some embodiments, the housing comprises a proximal sealing member, a distal sealing member, or any combination thereof. In some embodiments, the proximal sealing member and the distal sealing member comprise an elastomeric material configured to seal the housing within the ear of the subject. In some embodiments, the ultrasound transducer is electrically coupled to the one or more electric leads of the insert by the one or more wires. In some embodiments, the insert comprises a spacer structure configured to space the insert from the internal surface of the lumen of the housing. In some embodiments, the insert comprises a structure configured to releasably couple to the housing when inserted into the housing. In some embodiments, the structure comprises a groove, hole, or hook configured to snap to a structure of the housing. In some embodiments, the housing is partially or wholly vapor polished, aluminum coated, chrome coated, or any combination thereof. In some embodiments, the insert comprises an electrical coupling interface comprising an electro-mechanical structure configured to releasably couple with and be in electrically communication with a receptacle. In some embodiments, the electro-mechanical structure comprises one or more electrical pads adjacent a surface of one or more mechanical coupling interface. In some embodiments, the one or more mechanical coupling interface comprises a hook configured to couple with a clasp receptable.

In another aspect, the disclosure provides an otoscope, the otoscope comprising: a speculum and having a lumen therewithin and comprising a light conducting element, wherein a transmitted optical illumination is conducted by total internal reflection by the light conducting element, and wherein a reflected optical illumination is propagated through the lumen of the speculum; and one or more coupling portions which couple the transmitted optical illumination from a light source to the light conducting element, wherein the one or more coupling portions are shaped as a conic section. In some embodiments, the otoscope further comprises an insert, wherein the insert is configured to mechanically couple to the speculum. In some embodiments, the insert comprises a lens, an ultrasound transducer, one or more electrical leads electrically coupled to the ultrasound transducer, one or more wires electrically coupled to the one or more electrical leads and the ultrasound transducer, or any combination thereof. In some embodiments, the ultrasound transducer comprises a capacitive micromachined ultrasonic transducer. In some embodiments, the light conducting element comprises an ellipsoid shape. In some embodiments, the light conducting element is configured to be a parabolic mirror when light rays of the light source interact with the light conducting element. In some embodiments, the light conducting element comprises a launch point. In some embodiments, the light conducting element comprises at least two launch points. In some embodiments, the launch point comprises a geometry, wherein the geometry comprises: flat, circular, oval, trough, square, flat, or V-shaped. In some embodiments, the speculum comprises a proximal sealing member, a distal sealing member, or any combination thereof. In some embodiments, the proximal sealing member and the distal sealing member comprise an elastomeric material configured to seal the housing within the ear of the subject. In some embodiments, the ultrasound transducer is electrically coupled to the one or more electric leads of the insert by the one or more wires. In some embodiments, the insert comprises a spacer structure configured to space the insert from the internal surface of the lumen of the speculum. In some embodiments, the insert comprises a structure configured to releasably couple to the speculum when inserted into the speculum. In some embodiments, the structure comprises a groove, hole, or hook configured to snap to a structure of the speculum. In some embodiments, the speculum is partially or wholly vapor polished, aluminum coated, chrome coated, or any combination thereof. In some embodiments, the insert comprises an electrical coupling interface comprising an electro-mechanical structure configured to releasably couple with and be in electrical communication with a receptacle. In some embodiments, the electro-mechanical structure comprises one or more electrical pads adjacent a surface of the one or more mechanical coupling interface. In some embodiments, the one or more mechanical coupling interfaces comprise a hook configured to couple with a clasp receptable.

In another aspect, the disclosure provides a method of using an otoscope, the method comprising: directing optical illumination of a light source to one or more coupling portions, wherein the one or more coupling portions are shaped as a conic section; collimating the optical illumination using the one or more coupling portions; directing the optical illumination from the one or more coupling portions to a light conducting element, wherein the optical illumination propagates through the light conducting element by total internal reflection; and collecting reflected optical illumination from a target within a lumen of a housing, wherein the housing comprises a portion of a speculum of an otoscope. In some embodiments, the method further comprises directing pneumatic excitation toward the target. In some embodiments, the method further comprises directing ultrasound or illumination toward the target. In some embodiments, the method further comprises measuring a response of the target to the pneumatic excitation in a reflected ultrasound signal. In some embodiments, the method further comprises determining a state or condition of a subject based on the reflected optical illumination and the response.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure”, “FIG.” and “FIGS.” herein), of which:

FIG. 1 illustrates an exploded view of an example speculum tip, in accordance with embodiments.

FIG. 2A illustrates a side exterior view of an example speculum tip, in accordance with embodiments.

FIG. 2B illustrates a cross-section view of an example speculum tip, in accordance with embodiments.

FIG. 3A illustrates a view of a proximal end of an example speculum tip, in accordance with embodiments.

FIG. 3B illustrates an enlarged side view of a distal end of an example speculum tip, in accordance with embodiments.

FIG. 4A illustrates a view of distal end of an insert (e.g., a transducer mounted core (XMC)) of an example speculum tip, in accordance with embodiments.

FIG. 4B illustrates an enlarged isomorphic view of a distal end of an example speculum tip, in accordance with embodiments.

FIG. 5 illustrates another example of a speculum tip comprising a triangular launch point, in accordance with embodiments.

FIG. 6A illustrates a frustrated conic section comprising half an ellipsoid, in accordance with embodiments.

FIG. 6B illustrates an example of a petal shape as a sectioned frustrated conic section, in accordance with embodiments.

FIG. 7A shows an image of an illuminated hand polished speculum tip, in accordance with embodiments.

FIG. 7B shows an image of an illuminated vapor polished speculum tip, in accordance with embodiments.

FIG. 7C illustrates experimental data for optical transmission through an example speculum with changes in polishing of an exterior surface of the speculum.

FIGS. 8A-8B show images of illumination patterns of a commercial Welsch Allyn, speculum tip with solely a conical section, and a speculum tip with one or more petals taken at varying distances from the speculum exit tip.

FIG. 8C illustrates experimental data for intensity of the light transmitted through an example speculum with changes in coupling method of an optical source.

FIG. 9A illustrates a speculum tip with a fiber optic coupling, in accordance with embodiments.

FIG. 9B illustrates a speculum tip with a conical section, in accordance with embodiments.

FIG. 9C illustrates a speculum tip with a conical section in addition to one or more light conducting elements (“petals”), in accordance with embodiments.

FIG. 9D shows experimental data for intensity of the light transmitted through an example speculum with changes in geometry and/or method of light coupling from a light source.

FIGS. 10A-10D illustrate results of modeling a coupling angle of light into a speculum tip.

FIG. 11 illustrates results of modeling collimating characteristics of light coupled into a a light conducting element of a speculum tip, in accordance with embodiments.

FIG. 12A illustrates an image of light transmitted from example speculums tips at zero millimeter (mm) offset from the speculum tips.

FIG. 12B illustrates an image of light transmitted from example speculums tips at a twenty-five-millimeter offset from the speculum tips.

FIG. 12C shows an image of illuminated speculum tips for speculums tips with and without the ellipsoid light guide features (e.g., petals) attached to the speculum conical section.

FIG. 13 shows a computer system that is programmed or otherwise configured to implement methods provided herein.

FIG. 14 illustrates an exploded view of an example speculum tip, in accordance with embodiments.

FIG. 15A illustrates a side exterior view of an example speculum tip, in accordance with embodiments.

FIG. 15B illustrates a cross-section view of an example speculum tip, in accordance with embodiments.

FIG. 15C illustrates an isometric view of a proximal end of an example speculum tip, in accordance with embodiments.

FIG. 16A illustrates a view of distal end of an insert (e.g., an XMC) of an example speculum tip, in accordance with embodiments.

FIG. 16B illustrates an example of a speculum tip comprising a circular launch point, in accordance with embodiments.

FIG. 17A shows an image of a vapor polished speculum tip, in accordance with embodiments.

FIG. 17B shows an image of a chrome coated speculum tip, where the chrome coating is removed on launch and exit surfaces of the speculum tip, in accordance with embodiments.

FIG. 17C shows an image of a chrome coated speculum tip, where the chrome coating is removed on the speculum tip side walls near launch points and exit tip, in accordance with embodiments.

FIGS. 18A-18C show images of an experimental setup to measure light transmission and irradiance of the light emitted from a speculum tip, in accordance with embodiments.

FIG. 19 shows experimental results for measured optical irradiance transmitted through vapor polished and chrome coated speculum tips compared to electrical power of the illumination light emitting diode (LED) provided at the proximal end of the speculum tips.

FIG. 20 shows experimental results for measured irradiance falloff as a function of distance away from the distal tip of vapor polished and chrome coated speculum tips.

FIG. 21 shows measured experimental irradiance results over the course of five trials, where the irradiance was measured at ten-millimeter distal distance from vapor polished and chrome coated speculum tips.

FIG. 22A shows an image of an experimental setup for measuring the transmitted optical power and/or irradiance of a speculum tip without the presence of a tissue medium external to the speculum tip.

FIG. 22B shows an image of an experimental setup for measuring the transmitted optical power and/or irradiance of a speculum tip with the presence of a tissue medium external to the speculum tip.

FIG. 22C shows experimental results of absolute irradiance measured at the distal end of the vapor polished and chrome coated speculum tips with and without the presence of a tissue medium external to the speculum tip.

FIG. 23 shows images of the distal exit tip of vapor polished and chrome coated speculum measured at varying distances away from the distal exit tip.

FIG. 24A shows a schematic of a flat launch point geometry for speculum tips, in accordance with embodiments.

FIG. 24B shows a schematic of an oval launch point geometry for speculum tips, in accordance with embodiments.

FIG. 24C shows a schematic of a square launch point geometry for speculum tips, in accordance with embodiments.

FIG. 24D shows a schematic of a round launch point geometry for speculum tips, in accordance with embodiments.

FIG. 24E shows a schematic of a trough launch point geometry for speculum tips, in accordance with embodiments.

FIG. 24F shows a schematic of a V-shaped launch point geometry for speculum tips, in accordance with embodiments.

FIG. 25 shows experimental results of optical power at various distances from speculum exit tips for various launch point geometries.

FIG. 26A shows experimental simulated ray tracing for speculum tips with varying flat launch point geometries.

FIG. 26B shows experimental simulated ray tracing for speculum tips with varying oval launch point geometries.

FIG. 26C shows experimental simulated ray tracing for speculum tips with varying round launch point geometries.

FIG. 26D shows experimental simulated ray tracing for speculum tips with varying square launch point geometries.

FIG. 26E shows experimental simulated ray tracing for speculum tips with V-shaped launch point geometries.

FIG. 26F shows experimental simulated ray tracing for speculum tips with trough launch point geometries.

FIG. 27A shows images of various aluminum coated speculum tips, in accordance with embodiments.

FIG. 27B shows images of a patient's tympanic membrane illuminated by vapor polished and various chrome and aluminum coated speculum tips.

FIG. 28 shows a workflow diagram for a method of using an otoscope, in accordance with embodiments.

### DETAILED DESCRIPTION

The devices, otoscopes, specula, and methods of use and manufacture thereof as disclosed herein may address issues related to devices for measuring optical and ultrasound information. Embodiments of the present disclosure may improve upon the delivery of light and/or collection of light from a biological membrane, which may be characterized simultaneously with ultrasound excitation. The devices, otoscopes, specula, and methods of use and manufacture thereof as disclosed herein may address difficulties in the field regarding the alignment of device for measuring reflected ultrasound signals. In some case, present disclosure addresses issues in the field of otoscopy.

For example, surface characterization using an analysis of reflected ultrasound in the presence of a pneumatic excitation may be improved if the delivery of optical illumination, pneumatic excitation, and ultrasound signal is space efficient. For example, surface characterization using an analysis of reflected ultrasound in the presence of a pneumatic excitation may be improved if the measurement of reflected ultrasound signal and reflected optical illumination is space efficient.

For example, surface characterization using an analysis of reflected ultrasound in the presence of a pneumatic excitation may be improved if the ultrasound is directed at the surface at an angle that will result in ultrasound signal being returned to the transducer. Because ultrasound excitation is not visible to an eye, particularly the eye of a device operator, alignment of the ultrasound may be non-trivial. In one solution, a light source may be directed toward the surface to allow a user to better adjust an alignment of the device. The light source may be substantially aligned with the ultrasound propagation. In an ear, a user may align a light source within an ear canal to reflect light off the tympanic membrane. A good reflection may result in a “cone of light.” However, because a user may not look directly through the center of the lens and/or because a transducer may block the reflected light, the ultrasound and the light may not be propagating in the same direction.

The devices, otoscopes, specula, and methods of use and manufacture thereof as disclosed herein may be used in combination with for example devices and methods to characterize a ductile membrane, surface, and sub-surface properties such as those described in commonly owned U.S. Patent Publication 2020/0107813, U.S. Patent Publication 2018/0310917, and U.S. Patent Publication 2017/0014053, each of which is incorporated by reference in their entireties.

The devices, otoscopes, specula, and methods of use and manufacture thereof as disclosed herein may be used to characterize several biological tissues to provide a variety of diagnostic information. A biological tissue may comprise a patient organ. A speculum may be disposed within a bodily cavity to characterize a patient tissue. A patient organ or bodily cavity may comprise for example: a muscle, a tendon, a ligament, a mouth, a tongue, a pharynx, an esophagus, a stomach, an intestine, an anus, a liver, a gallbladder, a pancreas, a nose, a larynx, a trachea, lungs, kidneys, a bladder, a urethra, a uterus, a vagina, an ovary, a testicle, a prostate, a heart, an artery, a vein, a spleen, a gland, a brain, a spinal cord, a nerve, etc., or any combination thereof to name a few.

The devices, otoscopes, specula, and methods of use and manufacture thereof as disclosed herein may be used to characterize a tympanic membrane. For example, a tympanic membrane may be characterized to determine a condition of an ear, such as acute otitis media (AOM). A characterization that an ear exhibits AOM may include detection of the presence of effusion and characterization of the type of effusion as one of serous, mucoid, purulent, or combinations of these. In AOM, the middle ear effusion (MEE) may be induced by infective agents and may be thin or serous with viral infection and thicker and purulent with bacterial infection. Accordingly, determining various properties (shape or thickness of liquid, viscosity, or and/or other mechanical properties) of a fluid adjacent a tympanic membrane may provide information which may be used to characterize a membrane and/or provide a diagnosis to a patient.

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Certain inventive embodiments herein contemplate numerical ranges. When ranges are present, the ranges include the range endpoints. Additionally, every sub range and value within the range is present as if explicitly written out. The term “about” or “approximately” may mean within an acceptable error range for the particular value, which will depend in part on how the value is measured or determined, e.g., the limitations of the measurement system. For example, “about” may mean within 1 or more than 1 standard deviation, per the practice in the art. Alternatively, “about” may mean a range of up to 20%, up to 10%, up to 5%, or up to 1% of a given value. Where particular values are described in the application and claims, unless otherwise stated the term “about” meaning within an acceptable error range for the particular value may be assumed.

### Devices

A speculum, for example, one that houses a camera module may be shaped like a conic section funnel, which, in the extreme, mimics a hyperbolic rotation. These hyperbolic funnels are often used to teach physics students about orbits and gravity because of the properties they exhibit. It may be difficult to send something through one of these funnels without a lot of swirling and friction. It may be challenging to approach the center of the funnel on a radial path of the optical axis of the funnel, which may be advantageous to avoid swirling. In the optical case, excessive swirling may result in many rays turning around and exiting at the launch surface instead of the tip, which may result in light loss.

It may be advantageous to direct the rays from the LEDs onto a radial path of the optical axis of the funnel that leads to a direct route through the funnel and therefore avoiding swirling. A method which launches the rays down the radial direction of the funnel may accomplish this goal to a certain extent. However, since an LED is an extended source there may be limits to the efficiency of this scheme. Not all rays may be controlled well enough to make it through the funnel efficiently due, for example, to the etendue properties of the source.

It may be advantageous to provide a shape that is substantially similar to a section of an ellipsoid attached to the funnel shaped speculum. Such a shape may act as a section of an elliptical mirror and direct rays along the radial lines of the speculum thus improving the coupling efficiency between the light source and rays of light exiting the speculum tip for illumination.

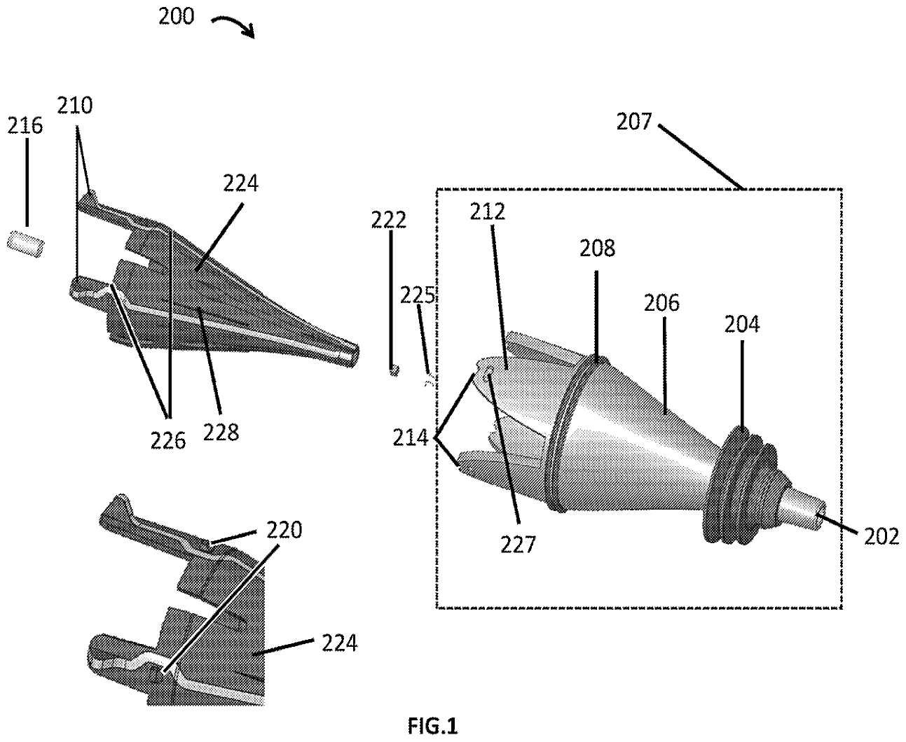

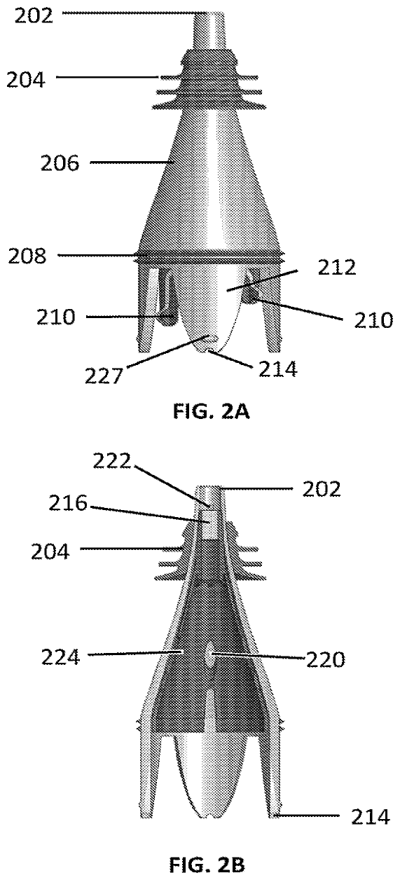

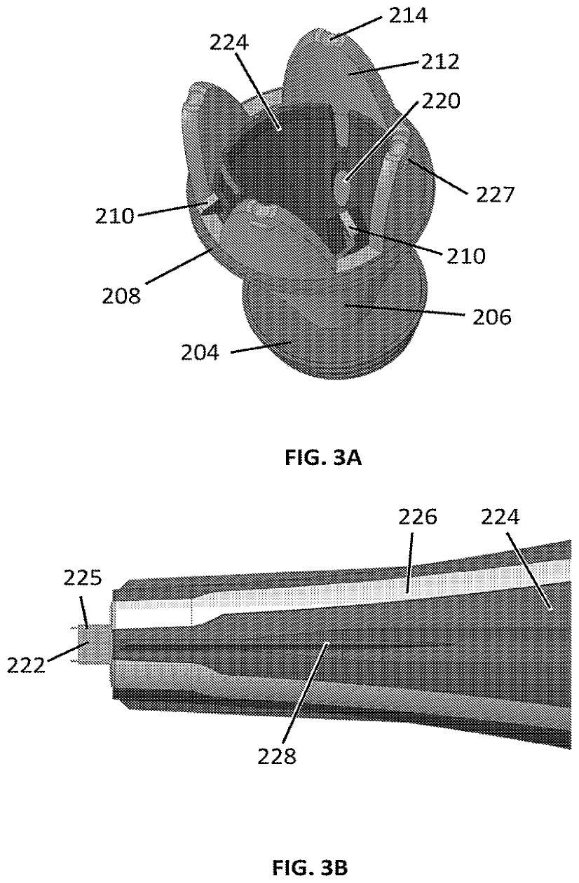

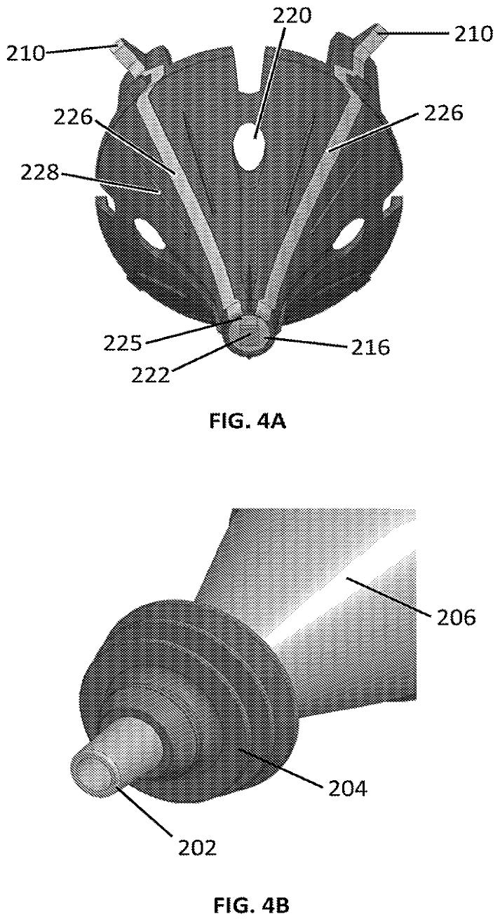

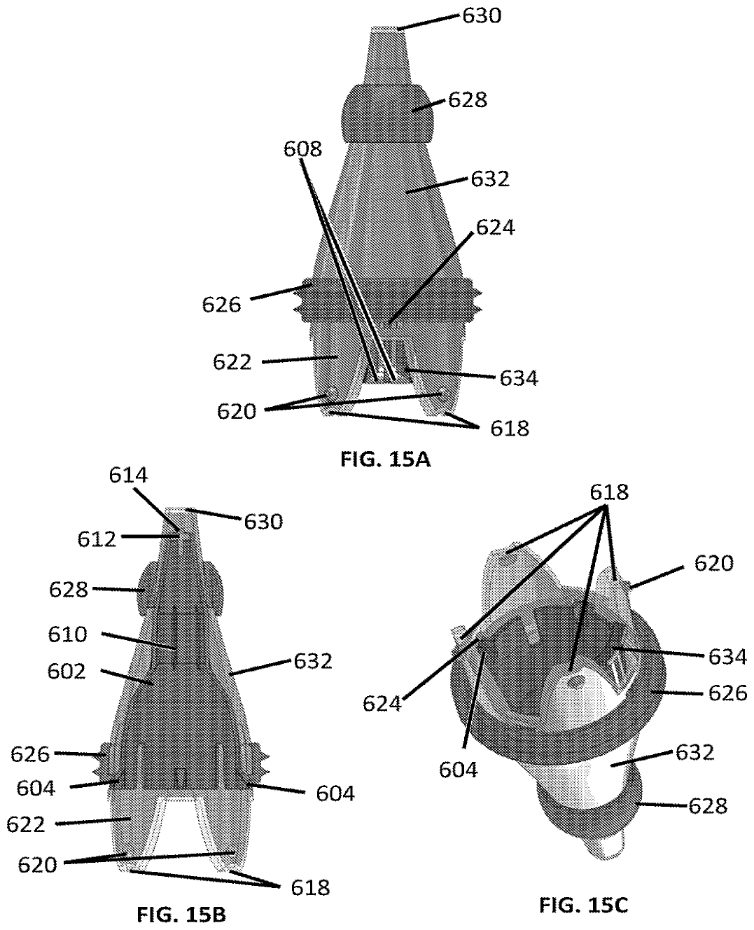

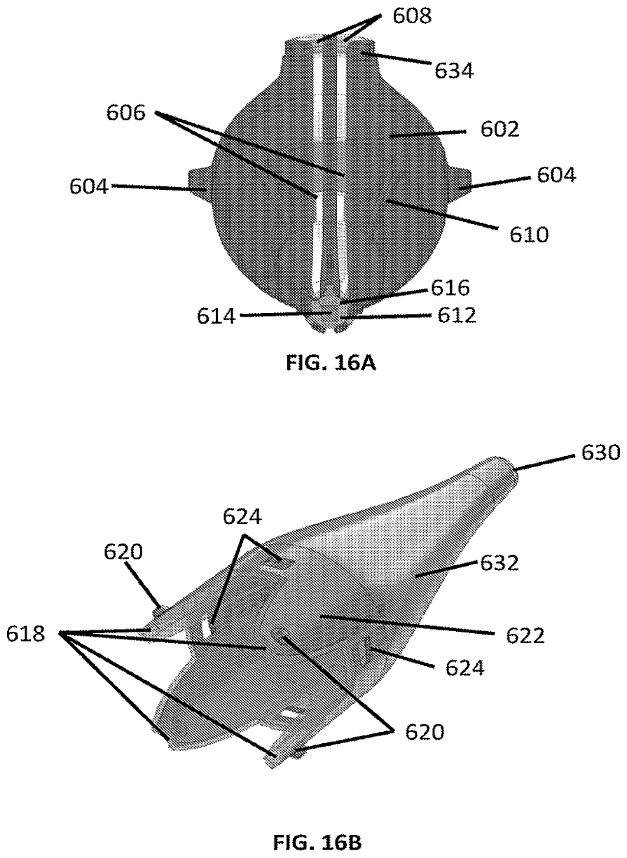

In some cases, the devices 200 and 600 described herein may comprise a speculum tip (207, 633), and insert (e.g., an XMC) (224, 602), as shown in FIGS. 1-5 and FIGS. 14-16B. In some instances, the speculum tip may be configured to illuminate a biological surface or a biological tissue of a patient or subject by redirecting a light source placed on the proximal end of the device to the distal exit tip (202, 630) of the speculum tip. In some instance, the speculum tip described elsewhere herein, may be configured such that light rays exiting the distal exit tip (202, 630) may reflect off a surrounding surface (e.g., an inner surface of a subject or patient's ear canal) and diffusely illuminate the subject or patient's tympanic membrane. In some cases, the diffusely reflected illumination may increase the total illumination on the subject's or patient's tympanic membrane, thereby enabling visualization and alignment of the device. In some cases, the speculum tip (207, 633), and insert (e.g., an XMC) (224, 602) may be made from the same material or different materials. The speculum tip may be manufactured from a material that transmits light from a light source (e.g., visible, near-infrared, etc.) with minimal loss. In some cases, the insert may be manufactured from a material that is electrically insulating and mechanically robust to hold the optical and ultrasound components for precise reproducible measurements. The biological surface may comprise a tympanic membrane of the patient or subject. In some cases, the illumination may comprise a uniform and/or diffuse illumination that evenly illuminates the biological surface and/or tissue. In some instances, the uniform and/or diffuse illumination may be generated by one or more light conducting element(s) (212,622), described elsewhere herein.

In some instances, the speculum tip (207, 633) may comprise a distal sealing member (204, 628), exit tip (202, 630), speculum conical section (206, 632), one or more light conducting elements (212, 622), a proximal sealing member (208, 626), one or more light source alignment features (227, 620), one or more launch points (214, 618) or any combination thereof, as seen in FIGS. 1-5 and FIGS. 14-16B.

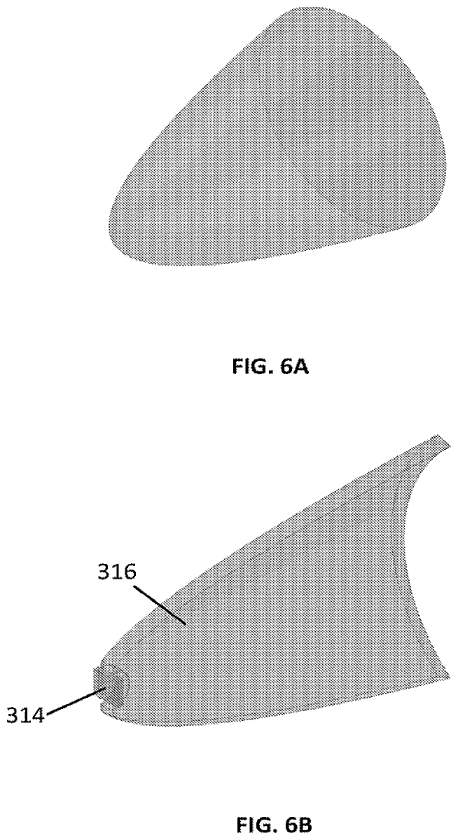

In some cases, the light conducting elements (212, 622) may comprise a portion of an ellipsoid shape 316, as seen in FIG. 6B. The contour and shape of the light conducting elements may collimate a point light source 314 or near point light source placed upon a distal launch point. In some cases, the light source may comprise a light emitting diode (LED). In some cases, the point source has an emission angle of up to about 50 degrees, about 60 degrees, about 70 degrees, about 80 degrees, about 90 degrees, about 100 degrees, about 120 degrees, about 130 degrees, about 140 degrees, about 150 degrees, about 160 degrees, about 170 degrees, or about 180 degrees. In some cases, the point source has an emission angle of at least about 50 degrees, about 60 degrees, about 70 degrees, about 80 degrees, about 90 degrees, about 100 degrees, about 120 degrees, about 130 degrees, about 140 degrees, about 150 degrees, about 160 degrees, about 170 degrees, or about 180 degrees.

As described elsewhere herein, the light conducting element's ellipsoid shape may prevent secondary and tertiary reflections of the coupled point source or near point source in manner causing the light rays to reflect back out of the light conducting element as light rays are coupled into a conical section, e.g., as shown in FIG. 6A. The light conducting elements collimate an input light source in a way that would prevent light rays exiting the light conducting elements with angled path with respect to the optical axis of the light conducting elements. Such angled paths would contribute to a loss in the total power of the light transmitted to the speculum tip. In some cases, the shape or contour of the light conducting elements may comprise a flower petal shape. Minimizing the angled reflection of exiting light with respect to the optical axis of the light conducting element may be particularly important when considering the coupling of the light conducting elements to a conical section as shown in FIG. 6A, and as described elsewhere herein. Rays may exit the “collimating” portion of the speculum (e.g., light conducting element or petal), and enter the conic section of the speculum with propagation directions that follow the radial planes of the conic section. In some cases, the exterior surface of the light conducting elements and/or the conic section may comprise a surface by which light rays propagating within the light conducting elements and/or the conical section will undergo total internal reflection. In some cases, the total internal reflection may direct light rays towards the radial path of the conic section and/or the light conducting element. In some cases, the refractive index difference between the material of the conical section and/or the light conducting segments and refractive index of the air or sample provided adjacent to the external surfaces of the conical section and/or the light conducting segments may alter total internal reflection. This principal may be utilized to increase total internal reflection e.g., by coating or polishing the surfaces or region thereof on the conical or light conducting elements. In some cases, the surface(s) or regions thereof may be coated with aluminum, chrome, platinum, gold, or any combination thereof. In some instances, the surface(s) or regions thereof may be vapor or hand polished. In some cases, the vapor or hand polished surfaces are intended to simulate or model the effect of molding or tooling the part when manufacturing the speculum tip.

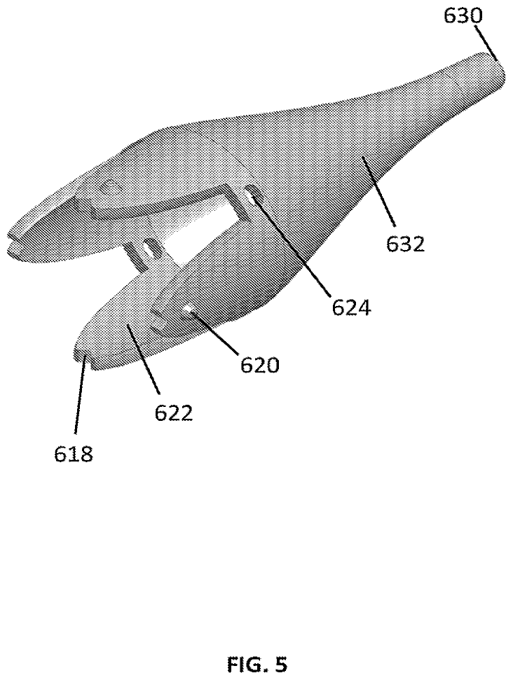

The light conducting elements (212, 622) may be adhered to, combined, molded together, or connected with the speculum conical section (206, 632) of the speculum tip. The intersection geometry between the light conducting elements and the speculum conical section may be a function of both production concerns (molding properties), and efficiency concerns. For example, FIG. 5 illustrates a cylindrical section of a petal light conducting element assembly with a draft to the one or more launch points (618) for molding efficiency. A triangular launch point may be efficient for the example shown in FIG. 5.

In some cases, the exit tip aperture distribution pattern may be affected by changes in the light conducting element conical section design. Changing the number of light conducting elements (i.e., petals) or the type of launch point geometry, described elsewhere herein, may change the illumination pattern at the exit tip. The examples of launch geometry and the number of petals provided herein may provide a sufficient illumination pattern while maintaining satisfactory efficiency.

In some cases, the light conducting elements (212, 622) may collimate or re-direct a light source such that the output light out of the speculum exit tip (202, 630) is at least 30% at least 40% at least 50% at least 60%, at least 70%, at least 80%, at least 90%, or at least 95% the output power of the light source coupled into the one or more launch points (214, 618). In some instances, the light source may comprise a light emitting diode, a surface light emitting diode, a super luminescent diode, a laser, a supercontinuum diode laser, pulsed laser, or any combination thereof. In some instances, the light source may comprise coupling optics between the light source and the one or more launch points (214, 618). In some cases, the coupling optics between the light source and the one or more launch points (214, 618) may comprise one or more lenses and/or index matching material between the one or more lenses and the one or more launch points (214, 618). In some cases, the one or more launch points (214, 618) may be optically coupled to the one or more light conducting elements (212, 622), the speculum conical section (206, 632), and/or the exit tip (202, 630) of the speculum tip. In some cases, the light conducting elements (212, 622) may comprise one or more light source alignment features (227, 620), where the one or more light source alignment features are configured to align placement of the light source with respect to the one or more launch points (214, 618). The one or more light source alignment features (227, 620) may protrude out and away from the surface of one or more light conducting elements.

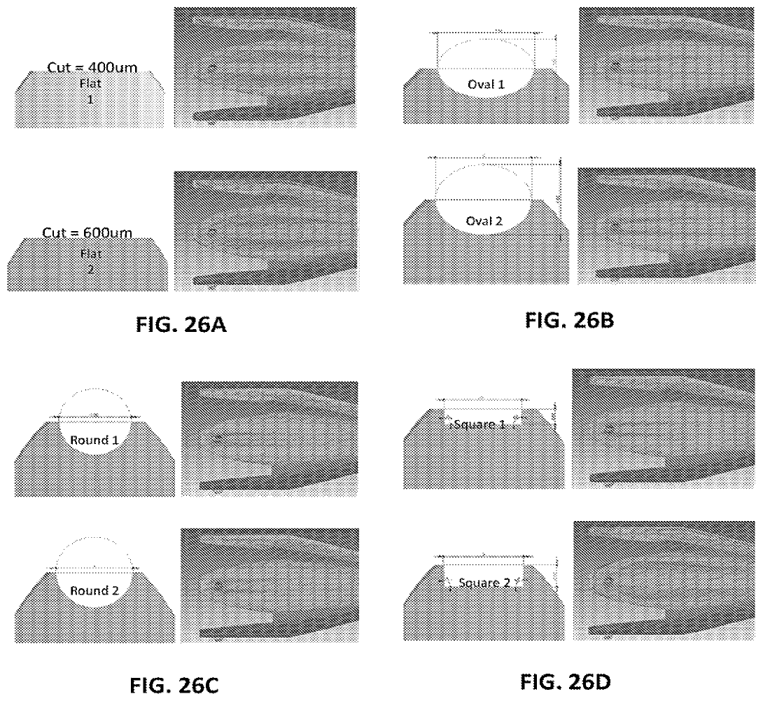

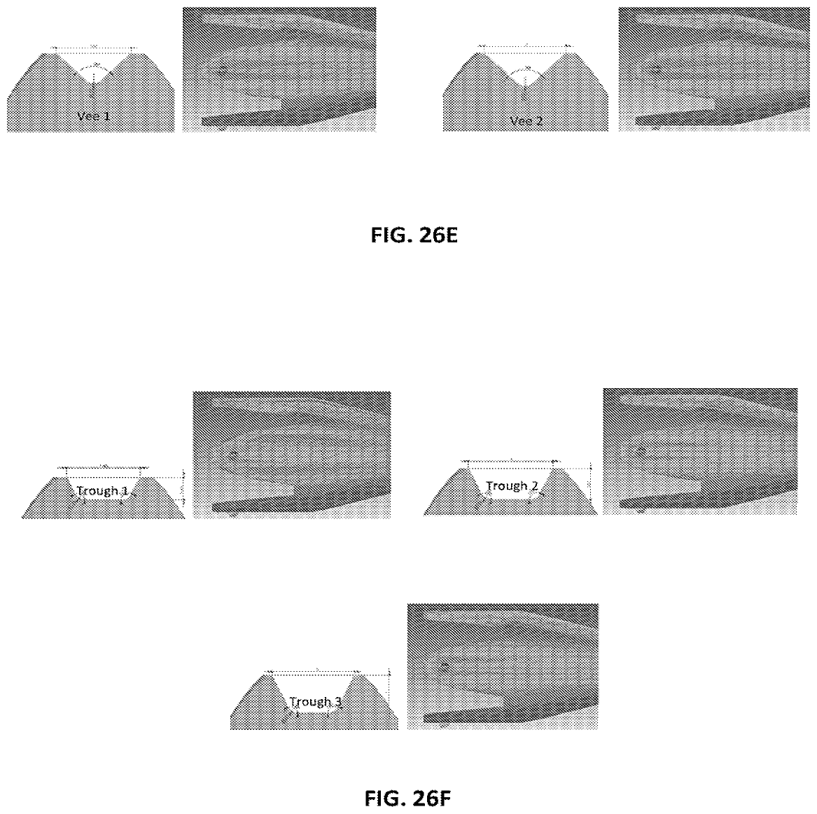

In some cases, the one or more launch points (214, 618) may be configured to interface and/or couple a light source into the one or more light conducting elements (212, 622). In some cases, the one or more launch points (214, 618) may comprise a geometry. The geometry may comprise flat, circular, oval, trough, triangular, square, or V-shaped geometry as seen in FIGS. 1-5, FIGS. 14-16B, and FIGS. 24A-24F. Each geometry may comprise varying dimensions that may affect the coupling efficiency of coupling the emitted light from the light source into the one or more light conducting elements (212, 622), as seen in ray tracing simulations of FIGS. 26A-26F.

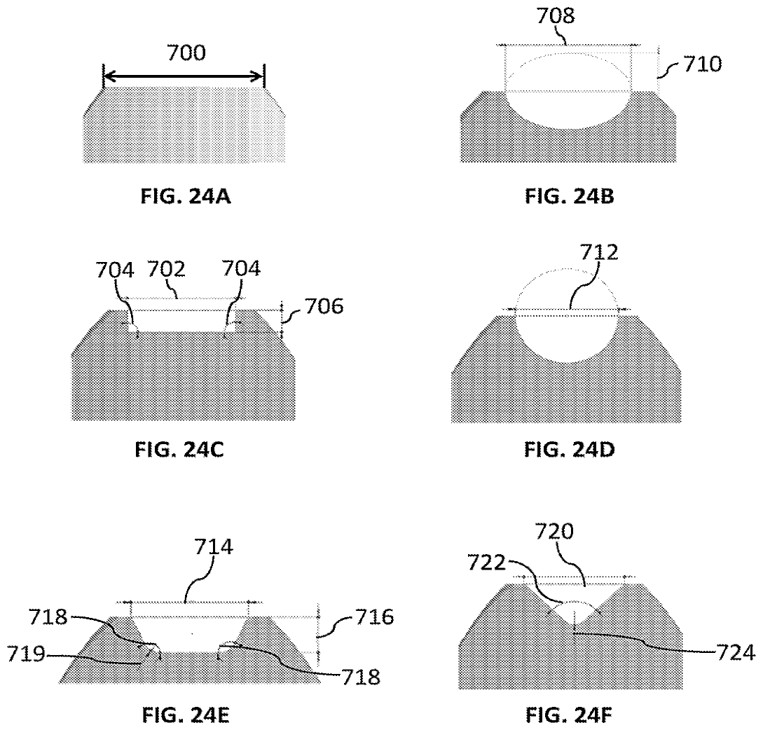

In some cases, the flat launch point geometry may comprise a length 700, as seen in FIG. 24A. In some cases, the length 700 may comprise about 200 micrometers (μm) to about 1,000 μm. In some cases, the length 700 may comprise about 200 μm to about 300 μm, about 200 μm to about 400 μm, about 200 μm to about 500 μm, about 200 μm to about 600 μm, about 200 μm to about 800 μm, about 200 μm to about 1,000 μm, about 300 μm to about 400 μm, about 300 μm to about 500 μm, about 300 μm to about 600 μm, about 300 μm to about 800 μm, about 300 μm to about 1,000 μm, about 400 μm to about 500 μm, about 400 μm to about 600 μm, about 400 μm to about 800 μm, about 400 μm to about 1,000 μm, about 500 μm to about 600 μm, about 500 μm to about 800 μm, about 500 μm to about 1,000 μm, about 600 μm to about 800 μm, about 600 μm to about 1,000 μm, or about 800 μm to about 1,000 μm. In some cases, the length 700 may comprise about 200 μm, about 300 μm, about 400 μm, about 500 μm, about 600 μm, about 800 μm, or about 1,000 μm. In some cases, the length 700 may comprise at least about 200 μm, about 300 μm, about 400 μm, about 500 μm, about 600 μm, or about 800 μm. In some cases, the length 700 may comprise at most about 300 μm, about 400 μm, about 500 μm, about 600 μm, about 800 μm, or about 1,000 μm.

In some cases, the circular launch point geometry, as seen in FIG. 24D, may comprise a diameter 712. In some cases, the diameter 712 may comprise about 0.5 millimeters (mm) to about 3 mm. In some cases, the diameter 712 may comprise about 0.5 mm to about 0.7 mm, about 0.5 mm to about 0.8 mm, about 0.5 mm to about 0.9 mm, about 0.5 mm to about 1 mm, about 0.5 mm to about 1.2 mm, about 0.5 mm to about 1.4 mm, about 0.5 mm to about 1.5 mm, about 0.5 mm to about 1.8 mm, about 0.5 mm to about 2 mm, about 0.5 mm to about 2.5 mm, about 0.5 mm to about 3 mm, about 0.7 mm to about 0.8 mm, about 0.7 mm to about 0.9 mm, about 0.7 mm to about 1 mm, about 0.7 mm to about 1.2 mm, about 0.7 mm to about 1.4 mm, about 0.7 mm to about 1.5 mm, about 0.7 mm to about 1.8 mm, about 0.7 mm to about 2 mm, about 0.7 mm to about 2.5 mm, about 0.7 mm to about 3 mm, about 0.8 mm to about 0.9 mm, about 0.8 mm to about 1 mm, about 0.8 mm to about 1.2 mm, about 0.8 mm to about 1.4 mm, about 0.8 mm to about 1.5 mm, about 0.8 mm to about 1.8 mm, about 0.8 mm to about 2 mm, about 0.8 mm to about 2.5 mm, about 0.8 mm to about 3 mm, about 0.9 mm to about 1 mm, about 0.9 mm to about 1.2 mm, about 0.9 mm to about 1.4 mm, about 0.9 mm to about 1.5 mm, about 0.9 mm to about 1.8 mm, about 0.9 mm to about 2 mm, about 0.9 mm to about 2.5 mm, about 0.9 mm to about 3 mm, about 1 mm to about 1.2 mm, about 1 mm to about 1.4 mm, about 1 mm to about 1.5 mm, about 1 mm to about 1.8 mm, about 1 mm to about 2 mm, about 1 mm to about 2.5 mm, about 1 mm to about 3 mm, about 1.2 mm to about 1.4 mm, about 1.2 mm to about 1.5 mm, about 1.2 mm to about 1.8 mm, about 1.2 mm to about 2 mm, about 1.2 mm to about 2.5 mm, about 1.2 mm to about 3 mm, about 1.4 mm to about 1.5 mm, about 1.4 mm to about 1.8 mm, about 1.4 mm to about 2 mm, about 1.4 mm to about 2.5 mm, about 1.4 mm to about 3 mm, about 1.5 mm to about 1.8 mm, about 1.5 mm to about 2 mm, about 1.5 mm to about 2.5 mm, about 1.5 mm to about 3 mm, about 1.8 mm to about 2 mm, about 1.8 mm to about 2.5 mm, about 1.8 mm to about 3 mm, about 2 mm to about 2.5 mm, about 2 mm to about 3 mm, or about 2.5 mm to about 3 mm. In some cases, the diameter 712 may comprise about 0.5 mm, about 0.7 mm, about 0.8 mm, about 0.9 mm, about 1 mm, about 1.2 mm, about 1.4 mm, about 1.5 mm, about 1.8 mm, about 2 mm, about 2.5 mm, or about 3 mm. In some cases, the diameter 712 may comprise at least about 0.5 mm, about 0.7 mm, about 0.8 mm, about 0.9 mm, about 1 mm, about 1.2 mm, about 1.4 mm, about 1.5 mm, about 1.8 mm, about 2 mm, or about 2.5 mm. In some cases, the diameter 712 may comprise at most about 0.7 mm, about 0.8 mm, about 0.9 mm, about 1 mm, about 1.2 mm, about 1.4 mm, about 1.5 mm, about 1.8 mm, about 2 mm, about 2.5 mm, or about 3 mm.

In some cases, the oval launch point geometry, as seen in FIG. 24B, may comprise a major 708 and minor diameter 710. In some instances, the major diameter 708 may comprise about 1 mm to about 2.6 mm. In some instances, the major diameter 708 may comprise about 1 mm to about 1.2 mm, about 1 mm to about 1.4 mm, about 1 mm to about 1.6 mm, about 1 mm to about 1.8 mm, about 1 mm to about 2 mm, about 1 mm to about 2.2 mm, about 1 mm to about 2.4 mm, about 1 mm to about 2.6 mm, about 1.2 mm to about 1.4 mm, about 1.2 mm to about 1.6 mm, about 1.2 mm to about 1.8 mm, about 1.2 mm to about 2 mm, about 1.2 mm to about 2.2 mm, about 1.2 mm to about 2.4 mm, about 1.2 mm to about 2.6 mm, about 1.4 mm to about 1.6 mm, about 1.4 mm to about 1.8 mm, about 1.4 mm to about 2 mm, about 1.4 mm to about 2.2 mm, about 1.4 mm to about 2.4 mm, about 1.4 mm to about 2.6 mm, about 1.6 mm to about 1.8 mm, about 1.6 mm to about 2 mm, about 1.6 mm to about 2.2 mm, about 1.6 mm to about 2.4 mm, about 1.6 mm to about 2.6 mm, about 1.8 mm to about 2 mm, about 1.8 mm to about 2.2 mm, about 1.8 mm to about 2.4 mm, about 1.8 mm to about 2.6 mm, about 2 mm to about 2.2 mm, about 2 mm to about 2.4 mm, about 2 mm to about 2.6 mm, about 2.2 mm to about 2.4 mm, about 2.2 mm to about 2.6 mm, or about 2.4 mm to about 2.6 mm. In some instances, the major diameter 708 may comprise about 1 mm, about 1.2 mm, about 1.4 mm, about 1.6 mm, about 1.8 mm, about 2 mm, about 2.2 mm, about 2.4 mm, or about 2.6 mm. In some instances, the major diameter 708 may comprise at least about 1 mm, about 1.2 mm, about 1.4 mm, about 1.6 mm, about 1.8 mm, about 2 mm, about 2.2 mm, or about 2.4 mm. In some instances, the major diameter 708 may comprise at most about 1.2 mm, about 1.4 mm, about 1.6 mm, about 1.8 mm, about 2 mm, about 2.2 mm, about 2.4 mm, or about 2.6 mm. In some cases, the minor diameter 710 may comprise about 1 mm to about 2.6 mm. In some cases, the minor diameter 710 may comprise about 1 mm to about 1.2 mm, about 1 mm to about 1.4 mm, about 1 mm to about 1.6 mm, about 1 mm to about 1.8 mm, about 1 mm to about 2 mm, about 1 mm to about 2.2 mm, about 1 mm to about 2.4 mm, about 1 mm to about 2.6 mm, about 1.2 mm to about 1.4 mm, about 1.2 mm to about 1.6 mm, about 1.2 mm to about 1.8 mm, about 1.2 mm to about 2 mm, about 1.2 mm to about 2.2 mm, about 1.2 mm to about 2.4 mm, about 1.2 mm to about 2.6 mm, about 1.4 mm to about 1.6 mm, about 1.4 mm to about 1.8 mm, about 1.4 mm to about 2 mm, about 1.4 mm to about 2.2 mm, about 1.4 mm to about 2.4 mm, about 1.4 mm to about 2.6 mm, about 1.6 mm to about 1.8 mm, about 1.6 mm to about 2 mm, about 1.6 mm to about 2.2 mm, about 1.6 mm to about 2.4 mm, about 1.6 mm to about 2.6 mm, about 1.8 mm to about 2 mm, about 1.8 mm to about 2.2 mm, about 1.8 mm to about 2.4 mm, about 1.8 mm to about 2.6 mm, about 2 mm to about 2.2 mm, about 2 mm to about 2.4 mm, about 2 mm to about 2.6 mm, about 2.2 mm to about 2.4 mm, about 2.2 mm to about 2.6 mm, or about 2.4 mm to about 2.6 mm. In some cases, the minor diameter 710 may comprise about 1 mm, about 1.2 mm, about 1.4 mm, about 1.6 mm, about 1.8 mm, about 2 mm, about 2.2 mm, about 2.4 mm, or about 2.6 mm. In some cases, the minor diameter 710 may comprise at least about 1 mm, about 1.2 mm, about 1.4 mm, about 1.6 mm, about 1.8 mm, about 2 mm, about 2.2 mm, or about 2.4 mm. In some cases, the minor diameter 710 may comprise at most about 1.2 mm, about 1.4 mm, about 1.6 mm, about 1.8 mm, about 2 mm, about 2.2 mm, about 2.4 mm, or about 2.6 mm.

In some instances, the square launch point geometry, as seen in FIG. 24C, may comprise a length 702, width 706, an internal angle 704. In some cases, the internal angle 704 may comprise an angle of about 90 degrees or about 91 degrees. In some instances, the length 702 may comprise about 1 mm to about 2.6 mm. In some instances, the length 702 may comprise about 1 mm to about 1.2 mm, about 1 mm to about 1.4 mm, about 1 mm to about 1.6 mm, about 1 mm to about 1.8 mm, about 1 mm to about 2 mm, about 1 mm to about 2.2 mm, about 1 mm to about 2.4 mm, about 1 mm to about 2.6 mm, about 1.2 mm to about 1.4 mm, about 1.2 mm to about 1.6 mm, about 1.2 mm to about 1.8 mm, about 1.2 mm to about 2 mm, about 1.2 mm to about 2.2 mm, about 1.2 mm to about 2.4 mm, about 1.2 mm to about 2.6 mm, about 1.4 mm to about 1.6 mm, about 1.4 mm to about 1.8 mm, about 1.4 mm to about 2 mm, about 1.4 mm to about 2.2 mm, about 1.4 mm to about 2.4 mm, about 1.4 mm to about 2.6 mm, about 1.6 mm to about 1.8 mm, about 1.6 mm to about 2 mm, about 1.6 mm to about 2.2 mm, about 1.6 mm to about 2.4 mm, about 1.6 mm to about 2.6 mm, about 1.8 mm to about 2 mm, about 1.8 mm to about 2.2 mm, about 1.8 mm to about 2.4 mm, about 1.8 mm to about 2.6 mm, about 2 mm to about 2.2 mm, about 2 mm to about 2.4 mm, about 2 mm to about 2.6 mm, about 2.2 mm to about 2.4 mm, about 2.2 mm to about 2.6 mm, or about 2.4 mm to about 2.6 mm. In some instances, the length 702 may comprise about 1 mm, about 1.2 mm, about 1.4 mm, about 1.6 mm, about 1.8 mm, about 2 mm, about 2.2 mm, about 2.4 mm, or about 2.6 mm. In some instances, the length 702 may comprise at least about 1 mm, about 1.2 mm, about 1.4 mm, about 1.6 mm, about 1.8 mm, about 2 mm, about 2.2 mm, or about 2.4 mm. In some instances, the length 702 may comprise at most about 1.2 mm, about 1.4 mm, about 1.6 mm, about 1.8 mm, about 2 mm, about 2.2 mm, about 2.4 mm, or about 2.6 mm. In some cases, the width 706 may comprise about 0.2 mm to about 1.2 mm. In some cases, the width 706 may comprise about 0.2 mm to about 0.3 mm, about 0.2 mm to about 0.4 mm, about 0.2 mm to about 0.5 mm, about 0.2 mm to about 0.6 mm, about 0.2 mm to about 0.8 mm, about 0.2 mm to about 1 mm, about 0.2 mm to about 1.2 mm, about 0.3 mm to about 0.4 mm, about 0.3 mm to about 0.5 mm, about 0.3 mm to about 0.6 mm, about 0.3 mm to about 0.8 mm, about 0.3 mm to about 1 mm, about 0.3 mm to about 1.2 mm, about 0.4 mm to about 0.5 mm, about 0.4 mm to about 0.6 mm, about 0.4 mm to about 0.8 mm, about 0.4 mm to about 1 mm, about 0.4 mm to about 1.2 mm, about 0.5 mm to about 0.6 mm, about 0.5 mm to about 0.8 mm, about 0.5 mm to about 1 mm, about 0.5 mm to about 1.2 mm, about 0.6 mm to about 0.8 mm, about 0.6 mm to about 1 mm, about 0.6 mm to about 1.2 mm, about 0.8 mm to about 1 mm, about 0.8 mm to about 1.2 mm, or about 1 mm to about 1.2 mm. In some cases, the width 706 may comprise about 0.2 mm, about 0.3 mm, about 0.4 mm, about 0.5 mm, about 0.6 mm, about 0.8 mm, about 1 mm, or about 1.2 mm. In some cases, the width 706 may comprise at least about 0.2 mm, about 0.3 mm, about 0.4 mm, about 0.5 mm, about 0.6 mm, about 0.8 mm, or about 1 mm. In some cases, the width 706 may comprise at most about 0.3 mm, about 0.4 mm, about 0.5 mm, about 0.6 mm, about 0.8 mm, about 1 mm, or about 1.2 mm.

In some cases, the trough launch point geometry, as seen in FIG. 24E, may comprise a length 714, width 716, internal fillet radii 719, and internal angle 718. In some instances, the internal angle 718 may comprise an angle of about 115 degrees, up to about 115 degrees, or at least about 115 degrees. In some cases, the length 714 may comprise about 1 mm to about 2.4 mm. In some cases, the length 714 may comprise about 1 mm to about 1.2 mm, about 1 mm to about 1.4 mm, about 1 mm to about 1.6 mm, about 1 mm to about 1.8 mm, about 1 mm to about 2 mm, about 1 mm to about 2.2 mm, about 1 mm to about 2.4 mm, about 1.2 mm to about 1.4 mm, about 1.2 mm to about 1.6 mm, about 1.2 mm to about 1.8 mm, about 1.2 mm to about 2 mm, about 1.2 mm to about 2.2 mm, about 1.2 mm to about 2.4 mm, about 1.4 mm to about 1.6 mm, about 1.4 mm to about 1.8 mm, about 1.4 mm to about 2 mm, about 1.4 mm to about 2.2 mm, about 1.4 mm to about 2.4 mm, about 1.6 mm to about 1.8 mm, about 1.6 mm to about 2 mm, about 1.6 mm to about 2.2 mm, about 1.6 mm to about 2.4 mm, about 1.8 mm to about 2 mm, about 1.8 mm to about 2.2 mm, about 1.8 mm to about 2.4 mm, about 2 mm to about 2.2 mm, about 2 mm to about 2.4 mm, or about 2.2 mm to about 2.4 mm. In some cases, the length 714 may comprise about 1 mm, about 1.2 mm, about 1.4 mm, about 1.6 mm, about 1.8 mm, about 2 mm, about 2.2 mm, or about 2.4 mm. In some cases, the length 714 may comprise at least about 1 mm, about 1.2 mm, about 1.4 mm, about 1.6 mm, about 1.8 mm, about 2 mm, or about 2.2 mm. In some cases, the length 714 may comprise at most about 1.2 mm, about 1.4 mm, about 1.6 mm, about 1.8 mm, about 2 mm, about 2.2 mm, or about 2.4 mm. In some instances, the width 716 may comprise about 0.5 mm to about 1.4 mm. In some instances, the width 716 may comprise about 0.5 mm to about 0.6 mm, about 0.5 mm to about 0.7 mm, about 0.5 mm to about 0.8 mm, about 0.5 mm to about 0.9 mm, about 0.5 mm to about 1 mm, about 0.5 mm to about 1.2 mm, about 0.5 mm to about 1.4 mm, about 0.6 mm to about 0.7 mm, about 0.6 mm to about 0.8 mm, about 0.6 mm to about 0.9 mm, about 0.6 mm to about 1 mm, about 0.6 mm to about 1.2 mm, about 0.6 mm to about 1.4 mm, about 0.7 mm to about 0.8 mm, about 0.7 mm to about 0.9 mm, about 0.7 mm to about 1 mm, about 0.7 mm to about 1.2 mm, about 0.7 mm to about 1.4 mm, about 0.8 mm to about 0.9 mm, about 0.8 mm to about 1 mm, about 0.8 mm to about 1.2 mm, about 0.8 mm to about 1.4 mm, about 0.9 mm to about 1 mm, about 0.9 mm to about 1.2 mm, about 0.9 mm to about 1.4 mm, about 1 mm to about 1.2 mm, about 1 mm to about 1.4 mm, or about 1.2 mm to about 1.4 mm. In some instances, the width 716 may comprise about 0.5 mm, about 0.6 mm, about 0.7 mm, about 0.8 mm, about 0.9 mm, about 1 mm, about 1.2 mm, or about 1.4 mm. In some instances, the width 716 may comprise at least about 0.5 mm, about 0.6 mm, about 0.7 mm, about 0.8 mm, about 0.9 mm, about 1 mm, or about 1.2 mm. In some instances, the width 716 may comprise at most about 0.6 mm, about 0.7 mm, about 0.8 mm, about 0.9 mm, about 1 mm, about 1.2 mm, or about 1.4 mm. In some cases, the internal fillet radii 719 may comprise a radius of about 0.1 mm to about 0.3 mm. In some cases, the internal fillet radii 719 may comprise a radius of about 0.1 mm to about 0.15 mm, about 0.1 mm to about 0.18 mm, about 0.1 mm to about 0.2 mm, about 0.1 mm to about 0.22 mm, about 0.1 mm to about 0.25 mm, about 0.1 mm to about 0.3 mm, about 0.15 mm to about 0.18 mm, about mm to about 0.2 mm, about 0.15 mm to about 0.22 mm, about 0.15 mm to about 0.25 mm, about 0.15 mm to about 0.3 mm, about 0.18 mm to about 0.2 mm, about 0.18 mm to about 0.22 mm, about 0.18 mm to about 0.25 mm, about 0.18 mm to about 0.3 mm, about 0.2 mm to about mm, about 0.2 mm to about 0.25 mm, about 0.2 mm to about 0.3 mm, about 0.22 mm to about 0.25 mm, about 0.22 mm to about 0.3 mm, or about 0.25 mm to about 0.3 mm. In some cases, the internal fillet radii 719 may comprise a radius of about 0.1 mm, about 0.15 mm, about mm, about 0.2 mm, about 0.22 mm, about 0.25 mm, or about 0.3 mm. In some cases, the internal fillet radii 719 may comprise a radius of at least about 0.1 mm, about 0.15 mm, about mm, about 0.2 mm, about 0.22 mm, or about 0.25 mm. In some cases, the internal fillet radii 719 may comprise a radius of at most about 0.15 mm, about 0.18 mm, about 0.2 mm, about mm, about 0.25 mm, or about 0.3 mm.

In some cases, the V-shaped launch point geometry, as seen in FIG. 24F, may comprise a length 720, internal angle 722, and internal fillet radius 724. In some cases, the internal angle 722 may comprise up to about 90 degrees, 90 degrees, or at least about 90 degrees. In some cases, the internal fillet radius 724 may comprise a radius of about 0.1 mm to about 0.3 mm. In some cases, the internal fillet radius 724 may comprise a radius of about 0.1 mm to about 0.15 mm, about 0.1 mm to about 0.18 mm, about 0.1 mm to about 0.2 mm, about 0.1 mm to about mm, about 0.1 mm to about 0.25 mm, about 0.1 mm to about 0.3 mm, about 0.15 mm to about 0.18 mm, about 0.15 mm to about 0.2 mm, about 0.15 mm to about 0.22 mm, about 0.15 mm to about 0.25 mm, about 0.15 mm to about 0.3 mm, about 0.18 mm to about 0.2 mm, about mm to about 0.22 mm, about 0.18 mm to about 0.25 mm, about 0.18 mm to about 0.3 mm, about 0.2 mm to about 0.22 mm, about 0.2 mm to about 0.25 mm, about 0.2 mm to about 0.3 mm, about 0.22 mm to about 0.25 mm, about 0.22 mm to about 0.3 mm, or about 0.25 mm to about 0.3 mm. the internal fillet radius 724 may comprise a radius of about 0.1 mm, about 0.15 mm, about 0.18 mm, about 0.2 mm, about 0.22 mm, about 0.25 mm, or about 0.3 mm. In some cases, the internal fillet radius 724 may comprise a radius of at least about 0.1 mm, about 0.15 mm, about 0.18 mm, about 0.2 mm, about 0.22 mm, or about 0.25 mm. In some cases, the internal fillet radius 724 may comprise a radius of at most about 0.15 mm, about 0.18 mm, about mm, about 0.22 mm, about 0.25 mm, or about 0.3 mm. In some cases, the length 720 may comprise about 1.2 mm to about 2.4 mm. In some cases, the length 720 may comprise about 1.2 mm to about 1.4 mm, about 1.2 mm to about 1.6 mm, about 1.2 mm to about 1.8 mm, about 1.2 mm to about 2 mm, about 1.2 mm to about 2.2 mm, about 1.2 mm to about 2.4 mm, about 1.4 mm to about 1.6 mm, about 1.4 mm to about 1.8 mm, about 1.4 mm to about 2 mm, about 1.4 mm to about 2.2 mm, about 1.4 mm to about 2.4 mm, about 1.6 mm to about 1.8 mm, about 1.6 mm to about 2 mm, about 1.6 mm to about 2.2 mm, about 1.6 mm to about 2.4 mm, about 1.8 mm to about 2 mm, about 1.8 mm to about 2.2 mm, about 1.8 mm to about 2.4 mm, about 2 mm to about 2.2 mm, about 2 mm to about 2.4 mm, or about 2.2 mm to about 2.4 mm. In some cases, the length 720 may comprise about 1.2 mm, about 1.4 mm, about 1.6 mm, about 1.8 mm, about 2 mm, about 2.2 mm, or about 2.4 mm. In some cases, the length 720 may comprise at least about 1.2 mm, about 1.4 mm, about 1.6 mm, about 1.8 mm, about 2 mm, or about 2.2 mm. In some cases, the length 720 may comprise at most about 1.4 mm, about 1.6 mm, about 1.8 mm, about 2 mm, about 2.2 mm, or about 2.4 mm.

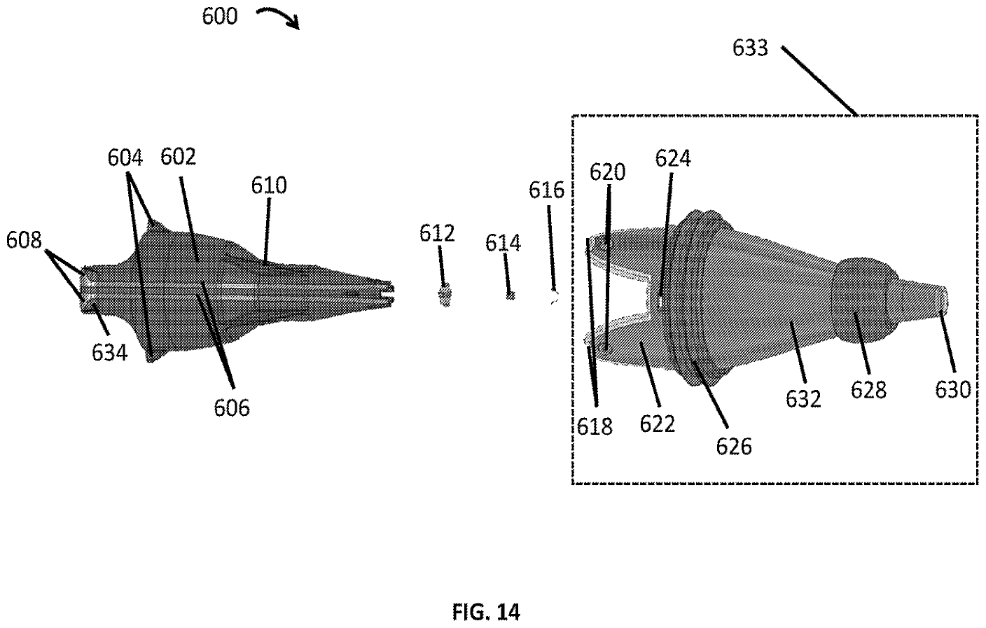

In some instances, the distal sealing member (204, 628) and/or the proximal sealing member (208, 626) may be comprised of a material that is configured to provide a seal between the device (200, 600) tip and a tubular cavity or orifice of a patient and/or subject that the speculum tip (207, 633) is inserted into. In some cases, the proximal sealing member 208 and/or the distal sealing member 204 may comprise one or more circular ring structures, as seen in FIGS. 1-5. In some instances, the proximal sealing member 626 may comprise one or more circular ring structures whereas the distal sealing member 628 may comprise a single elongated ring structure that follows the contour of the speculum tip conical segment, as seen in FIGS. 14-16B. The seal may be created to allow for pneumatic excitation of a biological surface or tissue and the recording of mechanical deformation of the biological surface and/or tissue. In some cases, the mechanical deformation of the biological surface and/or tissue may provide information regarding mechanical properties of the tissue, presence of abnormalities and/or heterogeneity of biological tissue mechanical properties, presence, or lack thereof fluid adjacent or in contact with the biological tissue and/or surface, or any combination thereof information of the biological surface and/or tissue. In some cases, measuring a presence or lack thereof fluid adjacent or in contact with the biological surface and/or tissue may provide diagnostic information to differentiate between acute otitis media, bacterial otitis media, viral otitis media, or any combination thereof conditions. In some instances, the mechanical deformation of the biological surface may be measured by an ultrasound transducer (222, 614).

In some cases, the insert (224, 602) may comprise an ultrasound transducer (222, 614) (e.g., a cMUT), one or more wires (225, 616) in electrical communication with the ultrasound transducer (222, 614) and the one or more leads of the insert (226, 606), a lens (216, 612), one or more mechanical coupling structures (220, 604) configured to couple to the speculum tip conical section (206, 632) at one or more mechanical fastening features (624), one or more electro-mechanical structures (210, 634) configured to interact with a corresponding receptacle, one or more mechanical support structures (228, 610) or any combination thereof as can be seen in FIGS. 1-5 and FIGS. 14-16B. In some cases, the one or more leads (226, 606) may comprise a trace of deposited material of gold or silver conductor adjacent the outer and/or inner surface of the inserter body. In some instances, the one or more leads may comprise a sheet of conductor that is inlaid into the outer surface of the insert (224, 602). In some cases, the electro-mechanical structure (210, 634) may comprise a pad contact, area, or region (608) of the electro-mechanical structure that is in electrical communication with the one or more leads (226, 606), one or more wires (225, 616), the ultrasound transducer (222, 614) or any combination thereof. In some instances, the electro-mechanical structure (210, 634) may be configured to mechanically and electrically couple to a receptacle of an otoscope to provide mechanical stability and to conduct electrical signals between the transducer and a base system to drive and receive signal from the ultrasound transducer. In some cases, the one or more mechanical support structures (228, 610) may comprise a rib, protrusion structure on the XMC insert (224, 602). The mechanical support structures may be configured to position the insert (224, 602) within the speculum conical section (206, 632) such that the light emission from the speculum exit tip (630, 202) and the ultrasound transducer (222, 614) and lens (216, 612) of the inserter are concentric.

In some cases, the one or more mechanical coupling structures (220, 604), may comprise a hook or protrusion that may to one or more mechanical fastening features (624). The one or more fastening features may comprise a corresponding feature that mates with the one or more mechanical coupling structures (220, 604) e.g., a cut out feature that a hook may snap into.

In some cases, the insert (224, 602) may comprise a lens (216, 612) coupled to the insert. In some cases, the lens may be in contact to or adjacent to the ultrasound transducer (222, 614). In some cases, the lens may be configured to focus reflected light of the light source off a biological surface and/or tissue for viewing by an operator of an otoscope fitted with the device (200, 600). In some cases, the lens may be placed proximal to the ultrasound transducer. In some instances, the lens may focus reflected light of the light source off a biological surface and/or tissue onto an image sensor, of the systems described elsewhere herein, that may convert the reflected light rays into a still image or video of the biological surface and/or tissue. In some cases, the lens may comprise an antireflective coating, where the antireflective coating transmits a first spectra and reflects a second spectra. In some instances, the first spectra may comprise the visible spectra and the second spectra may comprise an infrared spectrum.

In some instances, the one or more wires (225, 616) may be configured to transmit and/or relay electrical signals from the one or more leads (226, 606) to the ultrasound transducer (222, 614). In some cases, the one or more wires (225, 616) may connect to the one or more leads (226, 606) to the ultrasound transducer (222, 614) through wire bonding techniques.

In some cases, the device of the disclosure may comprise an ultrasound transducer (222, 614) that may electrically couple to the one or more leads via a surface mount pad of the ultrasound transducer (222, 614). In some cases, the ultrasound transducer (222, 614) may comprise a capacitive micromachined ultrasonic transducer (cMUT).

### Methods

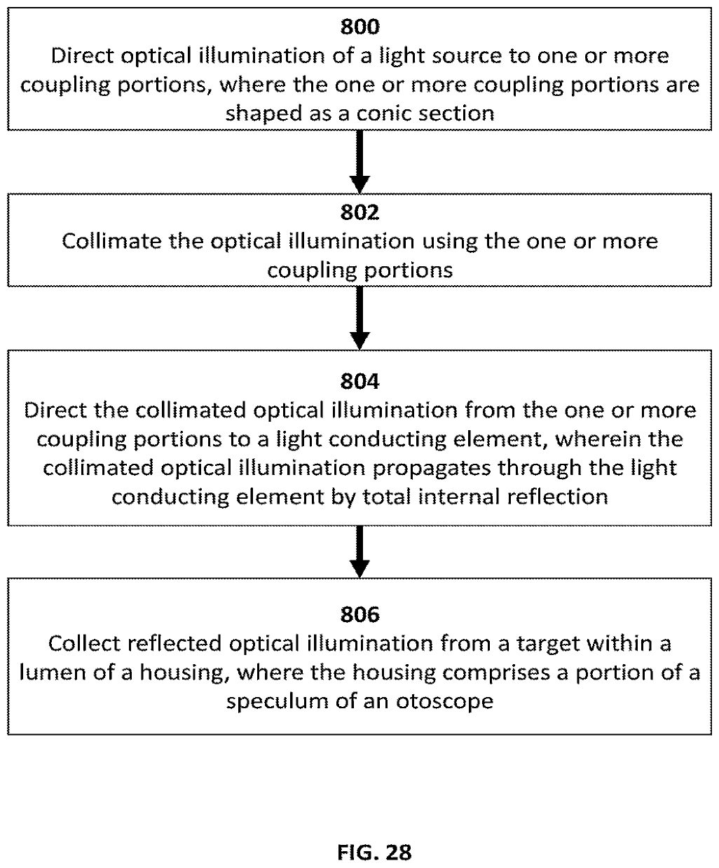

The present disclosure provides methods of using devices and related speculum tips, described elsewhere herein. In some cases, the method comprises the use of an otoscope with a speculum tip, as shown in FIG. 28. The method may comprise: (a) directing optical illumination of a light source to one or more coupling portions (i.e., launch points described elsewhere herein), where the one or more coupling portions are shaped as conic sections 800; (b) collimating the optical illumination using the one or more coupling portions 802; (c) directing the collimated optical illumination from the one or more coupling portions to a light conducting element (i.e., one or more light conducting elements or petals described elsewhere herein), where the collimated optical illumination propagates through the light conducting element by total internal reflection 804; and (d) collecting reflected optical illumination from a target within a lumen of a housing, where the housing comprises a portion of a speculum of an otoscope 806. In some embodiments, the method may further comprise directing pneumatic excitation toward the target. In some cases, the method may further comprise directing ultrasound or illumination toward the target. In some instances, the method may further comprise measuring a response of the target to the pneumatic excitation in a reflected ultrasound signal. In some cases, the method may further comprise determining a state or condition of a subject based on the reflected optical illumination and the response.

### Computer Systems

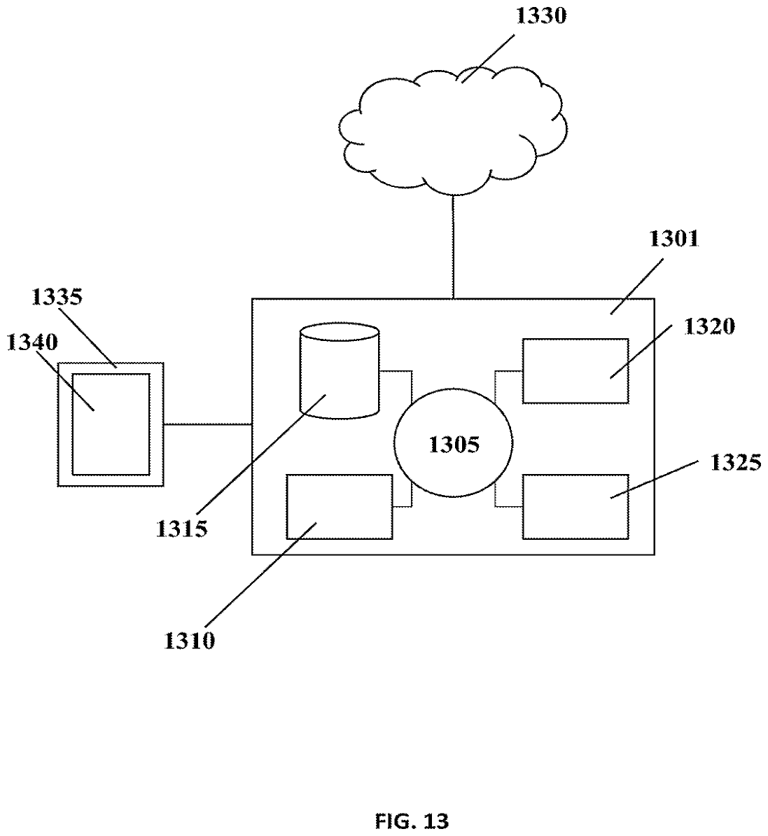

The present disclosure provides computer systems that are programmed to implement methods of the disclosure. FIG. 13 shows a computer system 1301 that is programmed or otherwise configured to control otoscopy systems and methods of the present disclosure. The computer system 1301 can regulate various aspects of an optical or ultrasound illumination system of an otoscope of the present disclosure, such as, for example, turning off or on various aspects of the device, analyzing data, acquiring data, providing driving signals to a light source and/or an ultrasound transducer etc. The computer system 1301 can be an electronic device of a user or a computer system that is remotely located with respect to the electronic device. The electronic device can be a mobile electronic device. The electronic device may be on board the otoscope.

The computer system 1301 includes a central processing unit (CPU, also “processor” and “computer processor” herein) 1305, which can be a single core or multi core processor, or a plurality of processors for parallel processing. The computer system 1301 also includes memory or memory location 1310 (e.g., random-access memory, read-only memory, flash memory), electronic storage unit 1315 (e.g., hard disk), communication interface 1320 (e.g., network adapter) for communicating with one or more other systems, and peripheral devices 1325, such as cache, other memory, data storage and/or electronic display adapters. The memory 1310, storage unit 1315, interface 1320 and peripheral devices 1325 are in communication with the CPU 1305 through a communication bus (solid lines), such as a motherboard. The storage unit 1315 can be a data storage unit (or data repository) for storing data. The computer system 1301 can be operatively coupled to a computer network (“network”) 1330 with the aid of the communication interface 1320. The network 1330 can be the Internet, an internet and/or extranet, or an intranet and/or extranet that is in communication with the Internet. The network 1330 in some cases is a telecommunication and/or data network. The network 1330 can include one or more computer servers, which can enable distributed computing, such as cloud computing. The network 1330, in some cases with the aid of the computer system 1301, can implement a peer-to-peer network, which may enable devices coupled to the computer system 1301 to behave as a client or a server.

The CPU 1305 can execute a sequence of machine-readable instructions, which can be embodied in a program or software. The instructions may be stored in a memory location, such as the memory 1310. The instructions can be directed to the CPU 1305, which can subsequently program or otherwise configure the CPU 1305 to implement methods of the present disclosure. Examples of operations performed by the CPU 1305 can include fetch, decode, execute, and writeback.

The CPU 1305 can be part of a circuit, such as an integrated circuit. One or more other components of the system 1301 can be included in the circuit. In some cases, the circuit is an application specific integrated circuit (ASIC).

The storage unit 1315 can store files, such as drivers, libraries, and saved programs. The storage unit 1315 can store user data, e.g., user preferences and user programs. The computer system 1301 in some cases can include one or more additional data storage units that are external to the computer system 1301, such as located on a remote server that is in communication with the computer system 1301 through an intranet or the Internet.

The computer system 1301 can communicate with one or more remote computer systems through the network 1330. For instance, the computer system 1301 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1301 via the network 1330.

Methods as described herein can be implemented by way of machine (e.g., computer processor) executable code stored on an electronic storage location of the computer system 1301, such as, for example, on the memory 1310 or electronic storage unit 1315. The machine executable or machine-readable code can be provided in the form of software. During use, the code can be executed by the processor 1305. In some cases, the code can be retrieved from the storage unit 1315 and stored on the memory 1310 for ready access by the processor 1305. In some situations, the electronic storage unit 1315 can be precluded, and machine-executable instructions are stored on memory 1310.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 1301, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1301 can include or be in communication with an electronic display 1335 that comprises a user interface (UI) 1340 for providing, for example, providing alignment information, providing diagnostic information, etc. Examples of UI's include, without limitation, a graphical user interface (GUI) i.e., a monitor screen or device display, and web-based user interface.

Methods and systems of the present disclosure can be implemented by way of one or more algorithms. An algorithm can be implemented by way of software upon execution by the central processing unit 1305. The algorithm can, for example, implement a method of characterizing a tympanic membrane.

### EMBODIMENTS

Numbered embodiment 1 comprises a speculum operable to be disposed within an ear of a subject, the speculum comprising: a housing comprising a light conducting element, wherein a transmitted optical illumination is conducted by total internal reflection via the light conducting element, wherein the housing has a lumen therewithin, and wherein the housing is configured to allow a reflected optical illumination to propagate through the lumen; and one or more coupling portions which couple the transmitted optical illumination from a light source to the light conducting element, wherein the one or more coupling portions are shaped as a conic section. Numbered embodiment 2 comprises the device of embodiment 1 further comprising an insert, wherein the insert is configured to be mechanically coupled to the housing. Numbered embodiment 3 comprises the device of embodiment 2, wherein the insert comprises a lens, an ultrasound transducer, one or more electrical leads electrically coupled to the ultrasound transducer, one or more wires electrically coupled to the one or more electrical leads and the ultrasound transducer, or any combination thereof. Numbered embodiment 4 comprises the device of embodiment 3 wherein the ultrasound transducer comprises a capacitive micromachined ultrasonic transducer. Numbered embodiment 5 comprises the device of any one of embodiments 1 to 4, wherein the light conducting element comprises an ellipsoid shape. Numbered embodiment 6 comprises the device of any one of embodiments 1 to 5, wherein the light conducting element is configured to be a parabolic mirror when light rays of the light source interact with the light conducting element. Numbered embodiment 7 comprises the device of any one of embodiments 1 to 6, wherein the light conducting element comprises a launch point. Numbered embodiment 8 comprises the device of any one of embodiments 1 to 6, wherein the light conducting element comprises one or more launch points. Numbered embodiment 9 comprises the device of embodiment 7 or 8, wherein the launch points comprise a geometry, wherein the geometry comprises: flat, round, oval, trough, square, or V-shaped. Numbered embodiment 10 comprises the device of embodiment 1, wherein the housing comprises a proximal sealing member, a distal sealing member, or any combination thereof. Numbered embodiment 11 comprises the device of any one of embodiments 1 to 10, wherein the proximal sealing member and the distal sealing member comprise an elastomeric material configured to seal the housing within the ear of the subject. Numbered embodiment 12 comprises the device of embodiment 3, wherein the ultrasound transducer is electrically coupled to the one or more electric leads of the insert by the one or more wires. Numbered embodiment 13 comprises the device as in embodiment 3 or 12, wherein the insert comprises a spacer structure configured to space the insert from the internal surface of the lumen of the housing. Numbered embodiment 14 comprises the device as in embodiments 3, 12, or 13, wherein the insert comprises a structure configured to releasably couple to the housing when inserted into the housing. Numbered embodiment 15 comprises the device of embodiment 14, wherein the structure comprises a groove, hole, or hook configured to snap to a structure of the housing. Numbered embodiment 16 comprises the device of any one of embodiments 1 to 14, wherein the housing is partially or wholly vapor polished, aluminum coated, chrome coated, or any combination thereof. Numbered embodiment 17 comprises the device of any one of embodiments 1 to 16, wherein the insert comprises an electrical coupling interface comprising an electro-mechanical structure configured to releasably couple with and be in electrically communication with a receptacle. Numbered embodiment 18 comprises the device of embodiments 17, wherein the electro-mechanical structure comprises one or more electrical pads adjacent a surface of one or more mechanical coupling interfaces. Numbered embodiment 19 comprises the device of embodiment 18, wherein the one or more mechanical coupling interfaces comprises a hook configured to couple with a clasp receptable.

Numbered embodiment 20 comprises an otoscope, the otoscope comprising: a speculum and having a lumen therewithin and comprising a light conducting element, wherein a transmitted optical illumination is conducted by total internal reflection by the light conducting element, and wherein a reflected optical illumination is propagated through the lumen of the speculum; and one or more coupling portions which couple the transmitted optical illumination from a light source to the light conducting element, wherein the one or more coupling portions are shaped as a conic section. Numbered embodiment 21 comprises the device of embodiment 20, further comprising an insert, wherein the insert is configured to mechanically couple to the speculum. Numbered embodiment 22 comprises the device of embodiment 21, wherein the insert comprises a lens, an ultrasound transducer, one or more electrical leads electrically coupled to the ultrasound transducer, one or more wires electrically coupled to the one or more electrical leads and the ultrasound transducer, or any combination thereof. Numbered embodiment 23 comprises the device of embodiment 22, wherein the ultrasound transducer comprises a capacitive micromachined ultrasonic transducer. Numbered embodiment 24 comprises the device of any one of embodiments 20 to 23, wherein said light conducting element comprises an ellipsoid shape. Numbered embodiment 25 comprises the device of any one of embodiments 20 to 24, wherein the light conducting element is configured to be a parabolic mirror when light rays of the light source interact with the light conducting element. Numbered embodiment 26 comprises the device of any one of embodiments 20 to 25, wherein the light conducting element comprises a launch point. Numbered embodiment 27 comprises the device of any one of embodiments 20 to 25, wherein the light conducting element comprises at least two launch points. Numbered embodiment 28 comprises the device as in embodiment 26 or 27, wherein the launch point comprises a geometry, wherein the geometry comprises: flat, round, oval, trough, square, or V-shaped. Numbered embodiment 29 comprises the device of any one of embodiments 20 to 28, wherein the speculum comprises a proximal sealing member, a distal sealing member, or any combination thereof. Numbered embodiment 30 comprises the device of embodiment 29, wherein the proximal sealing member and the distal sealing member comprise an elastomeric material configured to seal the housing within the ear of the subject. Numbered embodiment 31 comprises the device of any one of embodiments 22 to 27, wherein the ultrasound transducer is electrically coupled to the one or more electric leads of the insert by the one or more wires. Numbered embodiment 32 comprises the device of any one of embodiments 22 to 27 or 31, wherein the insert comprises a spacer structure configured to space the insert from the internal surface of the lumen of the speculum. Numbered embodiment 33 comprises the device of any one embodiments 22 to 27, 31, or 32, wherein the insert comprises a structure configured to releasably couple to the speculum when inserted into the speculum. Number embodiment 34 comprises the device of embodiment 33, wherein the structure comprises a groove, hole, or hook configured to snap to a structure of the speculum. Numbered embodiment 35 comprises the device of any one of embodiments 20 to 34, wherein the speculum is partially or wholly vapor polished, aluminum coated, chrome coated, or any combination thereof. Numbered embodiment 36 comprises the device of any one of embodiments 22 to 27 or 31 to 33, wherein the insert comprises an electrical coupling interface comprising an electro-mechanical structure configured to releasably couple with and be in electrically communication with a receptacle. Numbered embodiment 37 comprises the device of embodiment 36, wherein the electro-mechanical structure comprises one or more electrical pads adjacent a surface of one or more mechanical coupling interface. Numbered embodiment 38 comprises the device of embodiment 37, wherein the one or more mechanical coupling interface comprises a hook configured to couple with a clasp receptable.

Numbered embodiment 39 comprises a method of using an otoscope, the method comprising: directing optical illumination of a light source to one or more coupling portions, wherein the one or more coupling portions are shaped as a conic section; collimating the optical illumination using the one or more coupling portions; directing the optical illumination from the one or more coupling portions to a light conducting element, wherein the optical illumination propagates through the light conducting element by total internal reflection; and collecting reflected optical illumination from a target within a lumen of a housing, wherein the housing comprises a portion of a speculum of an otoscope. Numbered embodiment 40 comprises the method of embodiment 39, further comprising directing pneumatic excitation toward the target. Numbered embodiment 41 comprises the method as in embodiment 39 or 40, further comprising directing ultrasound or illumination toward the target. Numbered embodiment 42 comprises the method of embodiment 40, further comprising measuring a response of the target to the pneumatic excitation in a reflected ultrasound signal. Numbered embodiment 43 comprises the method of any one of embodiments 39 to 42, further comprising determining a state or condition of a subject based on the reflected optical illumination and the response.

### EXAMPLES

### Example 1: Determining Optimal Coupling Angles for Light Rays Entering the Conical Segment of the Speculum Tip

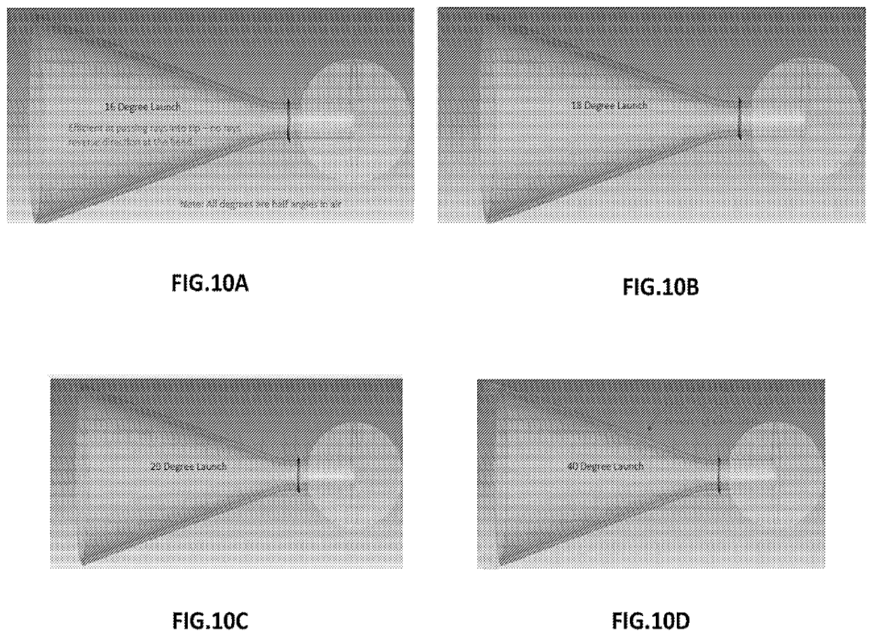

FIGS. 10A-10D show experimental ray tracing modeling data that was accomplished using Monte Carlo modeling methods. Various launch angles of 16 degrees (FIG. 10A), 18 degrees (FIG. 10B), 20 degrees (FIG. 10C), 40 degrees (FIG. 10D) were tested with a fixed conical section segment. To note, the launch angles are in degrees that are half angles in air. From the result of the ray tracing modeling experiment, a smaller launch angle e.g., at 16 degrees was more efficient at passing rays towards the tip of the conical section with none to minimal rays that exhibited back reflection off the conical section inner wall as was seen in larger launch degree angles. This finding suggests that rays of light entering at a very small angle with respect to the conical segment optical axis will exit with low to no insertion loss as they enter the conical section. The light conducting elements, described elsewhere herein, may address this point particularly with regards to coupling external light sources.

### Example 2: Simulated Ray Tracing of Petal Light Conducting Elements

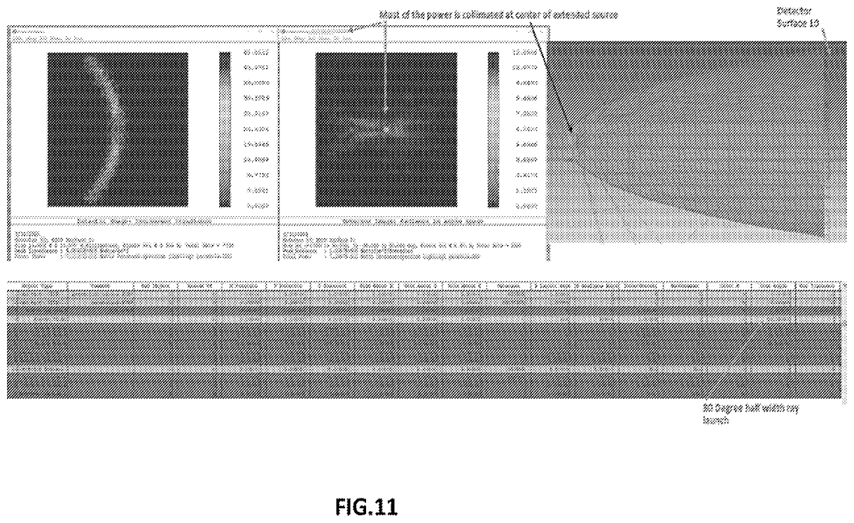

Ray tracing analysis of light coupling from a light source into and exiting the ellipsoid (i.e., “petal”) light conducting structure was conducted using Zemax, as seen in FIG. 11. From the ray tracing results, the “petal” ellipsoid structure of the light conducting element seems to function in a manner similar to a parabolic mirror to collimate the coupled emission of the light source concentrating rays parallel along the optical axis of the light conducting element. When taking a cross section through the light conducting element, light rays may be observed reflecting back and forth between the inner and outer surface of the light conducting element instead of back reflecting towards the direction of illumination source. By reducing and/or eliminating the back reflected light rays, the light conducting elements reducing the spiraling pattern trajectory light rays would otherwise take without going through the light conducting element.

### Example 3: Transmission Performance Compared Between Speculum Tips with and Without Light Conducting Elements

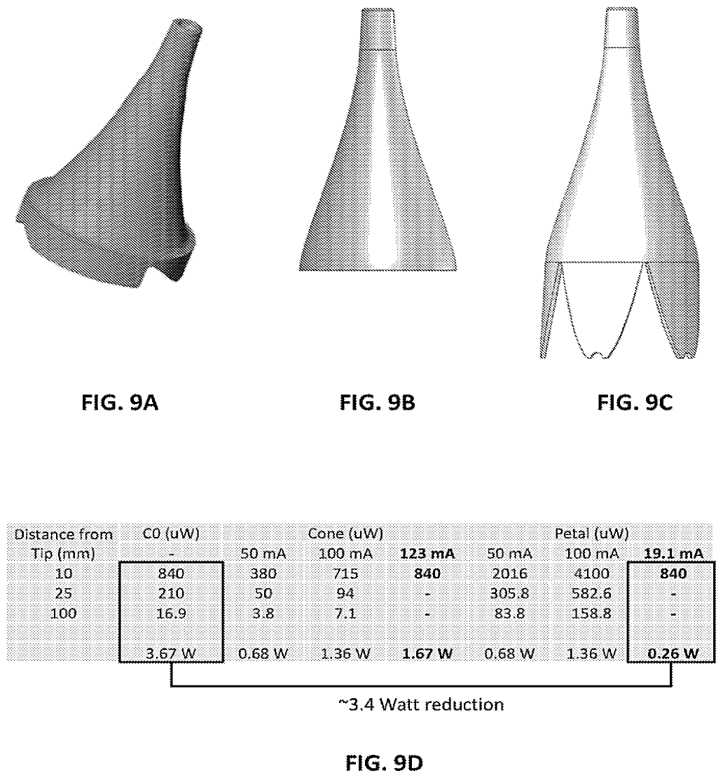

Optical power transmission was compared between a speculum tip with a conical section (FIG. 9B), speculum tip with the light conducting elements (i.e., “petals”) in addition to the conical section (FIG. 9C), and speculum tip with fiber optic coupling (FIG. 9A). The speculum tip with fiber optic coupling (“C0” referenced hereinafter) was designed to couple illumination light from a light source on a proximal end of the speculum tip through a plurality of 250 um diameter optical fibers. Optical power intensity measurements were made at varying displacement (10 mm, 25 mm, 100 mm) from the exit tip of the speculum tip and varying light source output current (50 mA, 100 mA, and 123 mA) for C0, a speculum tip with only the conical section, and a speculum tip with the conical section and the one or more light conducting elements, described elsewhere herein, the results of which are shown in the table of FIG. 9D. From the measured optical power, the speculum tip comprised of both the light conducting elements and the conical section could couple and transmit more optical power of the light source compared to the speculum tip with the conical section alone. In addition, the speculum tip comprised of the one or more light conducting elements in addition to the conical section shows significant efficiencies in measured illumination by producing comparable illumination of 840 uW of power at 19.1 mA as compared to the 840 uW of power transmitted by C0 at 734 mA, and 380 uW of optical power transmitted by the light conducting elements with the conical section alone of 380 uW at 50 mA. This result indicates that the combination of the conical section with one or more light conducting elements efficiently couples a distal light source to a proximal reduced geometry.

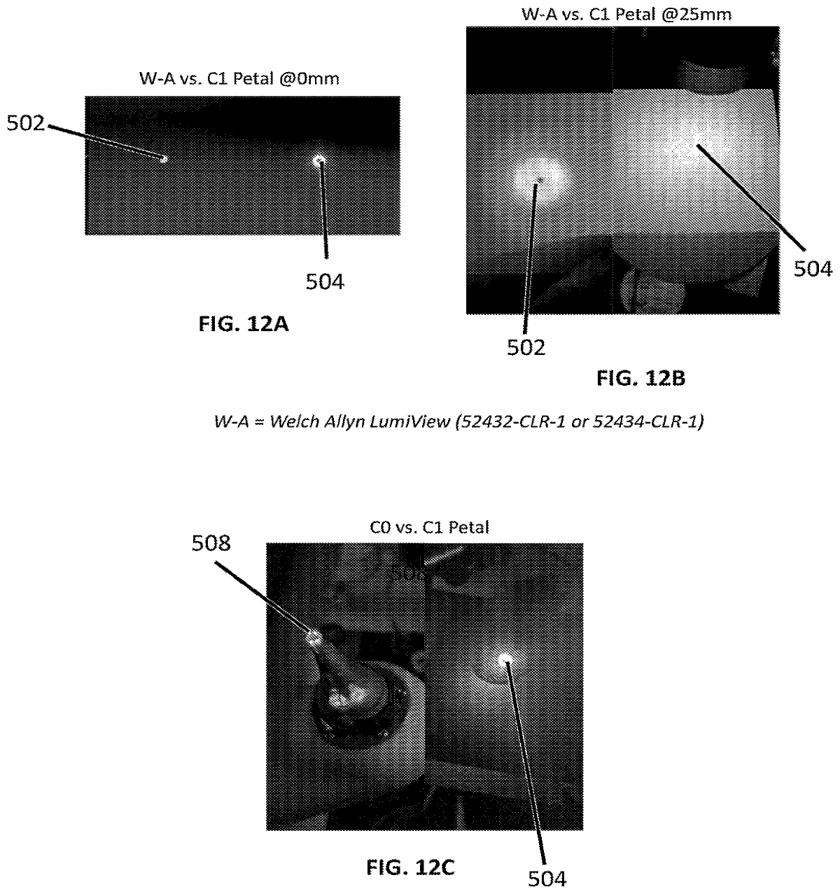

An experiment showing visual brightness variation between speculum tips C0 508, and the conical section with the one or more light conducting elements (“petal”) 504 may be seen in FIG. 12C. A five times increase in brightness at the same light source drive current was measured and observed in the image shown.

### Example 4: Comparing Hand Polished and Vapor Polished Speculum Tips

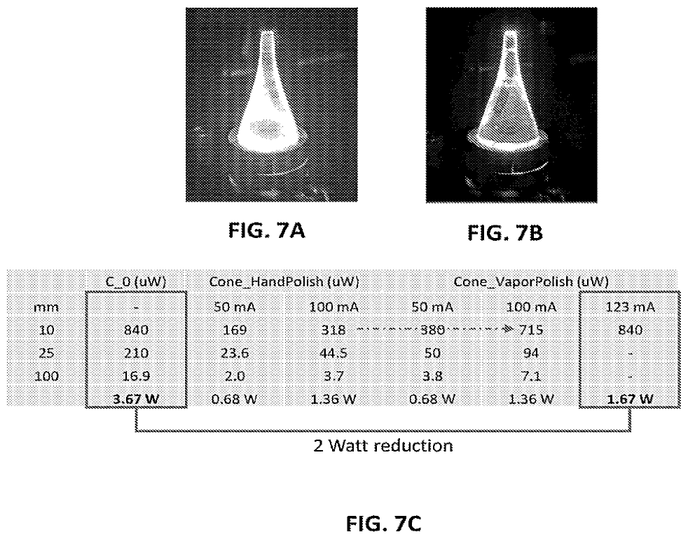

Speculum tips were hand polished (FIG. 7A) and vapor polished (FIG. 7B) to determine if post processing of the speculum tip would improve transmission from the light source through the one or more light conducting elements and conical section of the speculum tip. Optical power intensity measurements were made at varying displacement (10 mm, 25 mm, 100 mm) and varying light source output current (50 mA, 100 mA, and 123 mA) for a clinical prototype C_0, described in Example 3, hand polished, and vapor polished speculum tips, the results of which are summarized in FIG. 7C. The clinical prototype speculum tip (C_0) included four 250 μm outer diameter fibers coupled to the inner lumen of the speculum tip. The four 250 μm fibers were but coupled on the proximal end of the speculum tip to four corresponding 500 um outer diameter optical fibers providing illumination. The hand polish and vapor polished speculum tips comprised a waveguide conical section, described elsewhere herein, configured to but couple to a light source on the proximal end of the speculum tip, as shown in FIGS. 7A-7B. From the measurement results, the vapor polished speculum tip outperformed the hand polished speculum tip with a difference of approximately 400 uW of power at 10 mm displacement from the exit tip at 100 mA light source current, 46 uW of power at 25 mm displacement from the exit tip at 100 mA, and 4 uW of power at 100 displacement from the exit tip.

### Example 5: Effects of a Biological Tissue Placed Adjacent to Speculum Tips of Various Post Processing Approaches

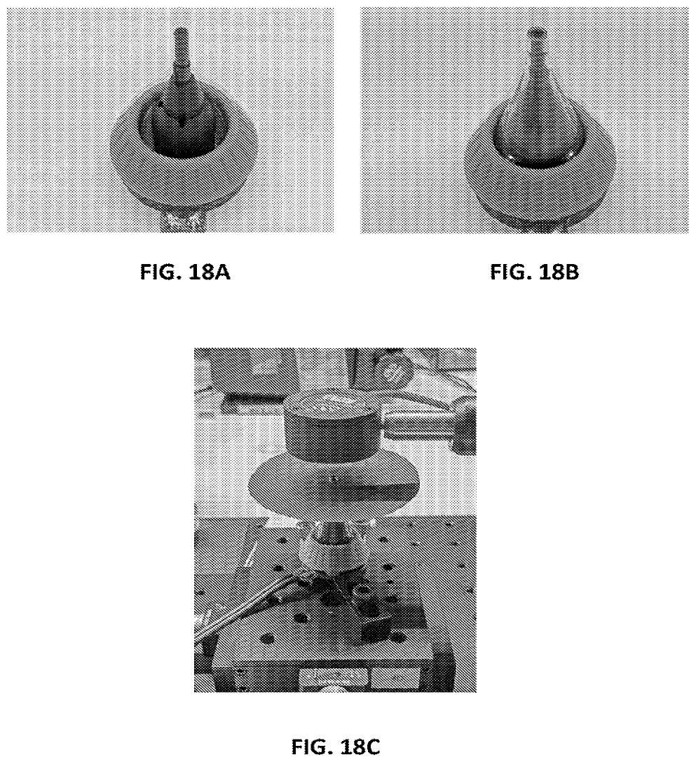

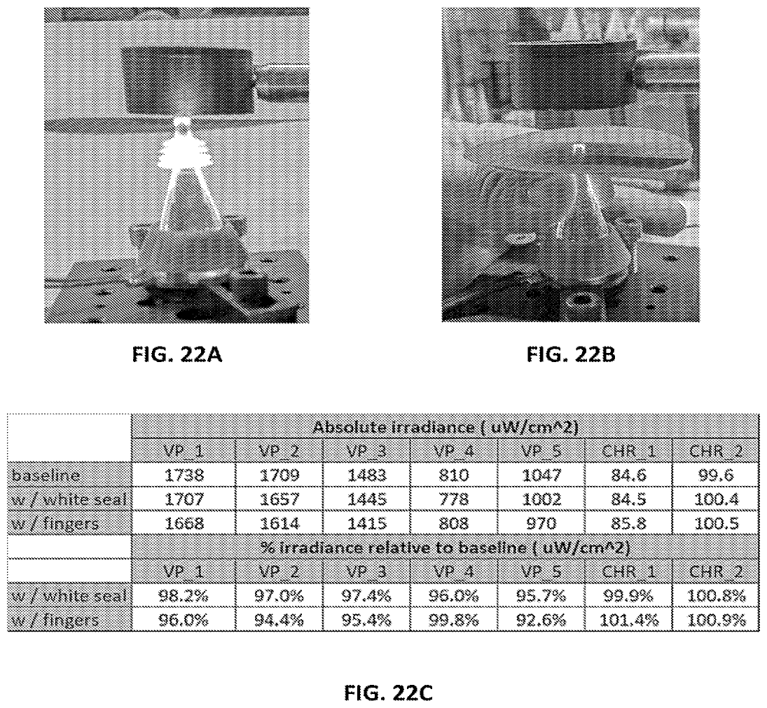

The effect of an adjacent biologic surface held against an outer surface of the speculum tip on measured absolute irradiance (μW/cm²) and % irradiance relative to baseline (μW/cm²) was determined, as shown FIGS. 22A-C. For purposes of this experiment, both vapor polished (VP_1 to VP_5) and chrome coated (CHR_1 and CHR_2) speculum tips were compared when a biological surface of a finger or a white seal was placed adjacent to the outer surface of the speculum tip conical section. A white seal for purposes of this experiment was a piece of white colored elastomeric material configured to mimic a human biological surface e.g., human skin, that would allow the measurement of light “bleed” or refraction out light of the speculum tip (i.e., light conduit or light pipe) when such a material was present adjacent to the surface of the speculum tip. To acquire irradiance measurements, each speculum tip distal surface was but coupled to combined light source otoscope receptacle interface as shown in FIG. 18A. Prior to placing each speculum tip into the light source otoscope receptacle a 2.5 mm pin was placed on the distal tip of the otoscope receptable to prevent the detection of stray light from the proximal light source traveling through the center of the speculum tip, seen in FIG. 18A. After seating the speculum tip into the light source otoscope receptacle, as seen in FIG. 18B, a paper baffle was placed around the proximal tip of the speculum to further block stray light from the light source to confine measurements to light transmitted by the speculum tip shown in FIG. 18C, and FIGS. 22A-B. Measurements of emitted light from the speculum tips was measured using a Model 818-SL silicon detector head with 1 cm 2 area and Model 1830-C power meter with wavelength calibration set for 550 nm. The light source utilized for this measurement was driven by a HP E3631A DC power supply in current control mode. From the results, shown in FIG. 22C, an approximate 3-4% decrease in efficiency was observed when a white seal was placed in contact with the outer surface of vapor polished speculum tips and an approximate 5-7% decrease in efficiency when the speculum tip is pressed between a thumb and forefinger near the tip as shown in FIG. 22B. No change in efficiency was observed when the chrome coated speculum tips were subjected to the white seal or thumb and forefinger as provided to the vapor polished speculum tips.

### Example 6: Comparison of Illumination Performance of Vapor Polished and Chrome Coated Speculum Tips

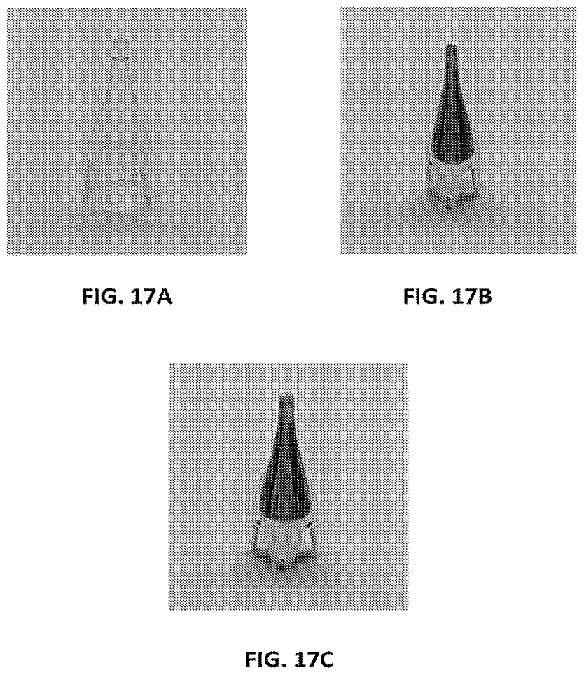

Illumination performance between vapor polished FIG. 17A and chrome coated speculum tips (FIGS. 17B-C) was conducted to determine any differences between the two types of speculum tip finishing processes. A representative vapor polished speculum tip is shown in FIG. 17A, where the vapor polish was applied to all surfaces of the speculum tip except the light source launch points and speculum exit tip, described elsewhere herein. The vapor polished speculum tips will be referenced as VP_1 to VP_15 in corresponding figures. The chrome coated speculum tips were comprised of variable regions of chrome coating. FIG. 17B shows a chrome coated speculum tip where chrome coating was applied to all interior and exterior surfaces of the speculum tip without coating the multiple light source launch points and the distal exit tip of the speculum, described elsewhere herein. Such chrome coated speculum tips will be referenced with the identifier CHR_1 through CHR_3 in corresponding figures. FIG. 17C shows a chrome coated speculum tip where chrome coating was applied to all interior and exterior surface of the speculum tip without coating the multiple light source launch points, distal exit tip of the speculum, and a mask region on the side walls near the distal exit tip of the speculum. Such chrome coated speculum tips will be referenced with the identifier CwM_1 to CwM_3 in the results referenced in corresponding figures.

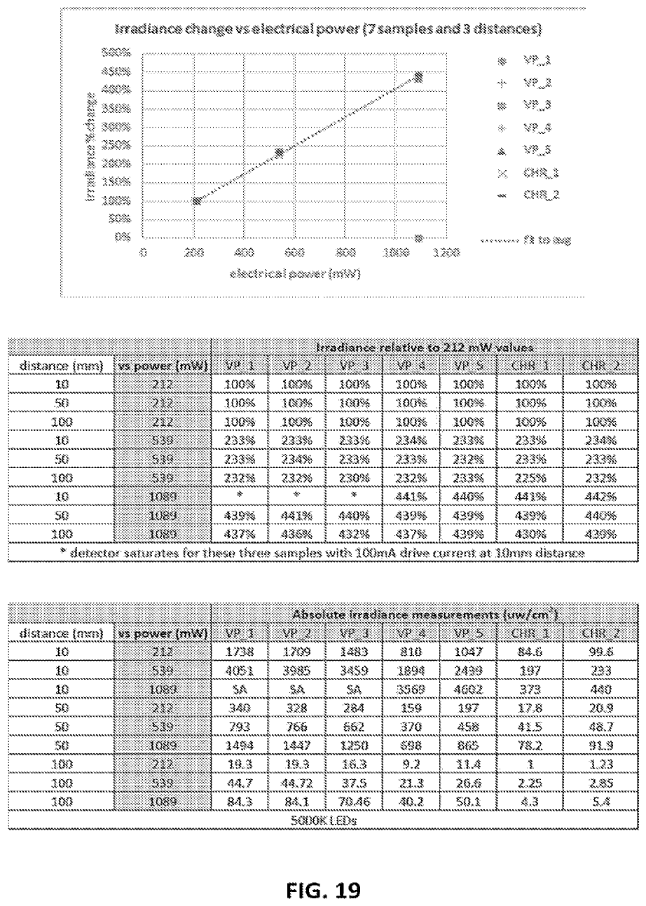

Each speculum tip was placed into an experimental illumination receptacle as outlined in Example 5 to prevent stray light from adding noise to the illumination measurement. Illumination measurements (FIGS. 19-21) were made by both varying the current of the light source (LED light source in current control power mode) and distance between the detector and the distal exit tip of the speculum.

FIG. 19 shows the measured irradiance percent change vs. electrical power for the speculum tips at three detector distances (5, 10, and 15 mm) from the distal exit tip of the speculums. From the results shown in FIG. 19 optical irradiance and electrical power share a linear relationship and is similar across the various types of speculum tips and is independent of detector measurement distance.

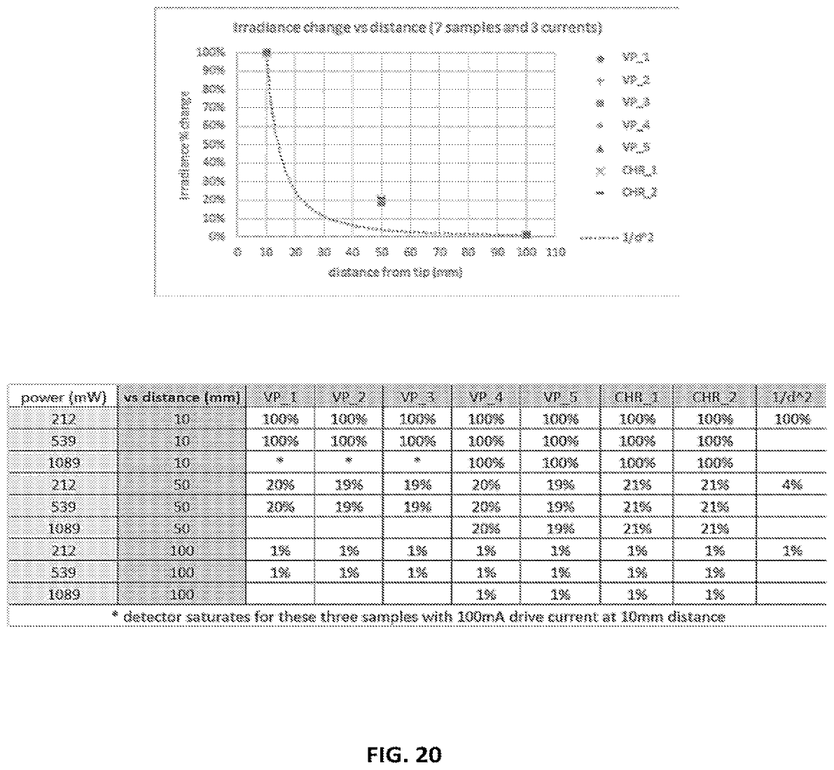

FIG. 20 shows the measured irradiance percent change vs. detector distance from the distal exit tip of the speculum. From the results, irradiance falloff as a function of detector measurement distance is the same for both vapor polished and chrome coated speculum tips. Additionally, the relationship of irradiance falloff as a function of detector measurement distance is independent of electrical power.

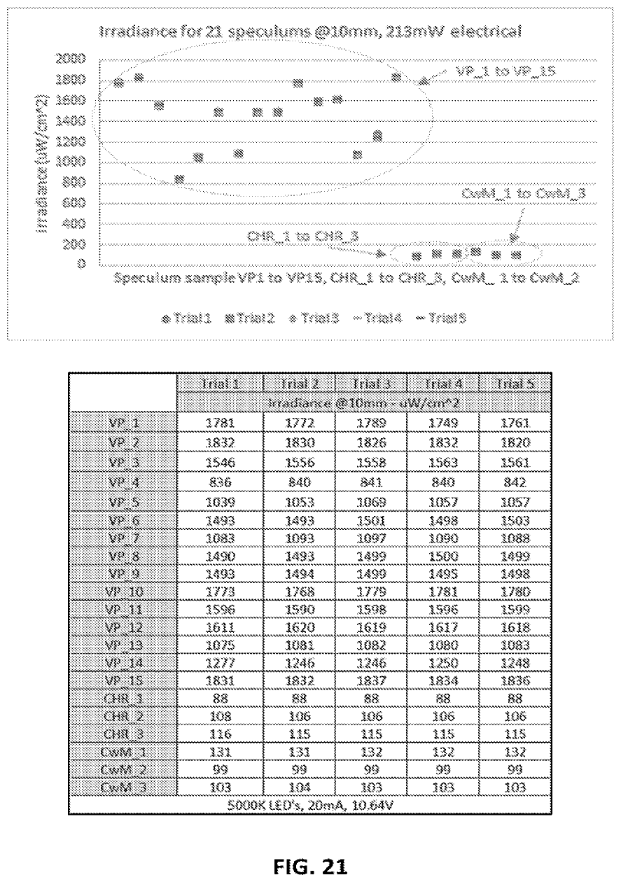

FIG. 21 shows measured irradiance for the various types of speculum tips at a 10 mm distance from the distal exit tip of the speculum. Irradiance was measured over five temporally distinct trials. From the results shown in FIG. 21, the best performing vapor polished speculum tip performed with approximately sixteen times efficiency than either variety of chrome coated speculum tips.

### Example 7: Simulated Ray Tracing and Illumination Transmission Efficiencies of Launch Point Geometries

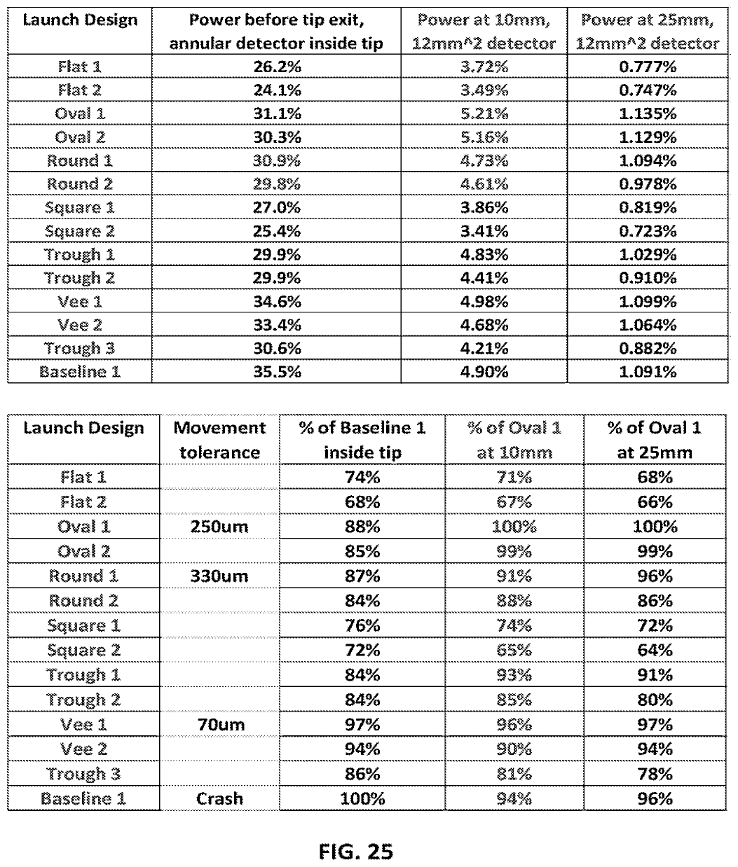

Simulated ray tracing and optical illumination transmission was completed for various launch point geometries, described elsewhere herein, shown in FIGS. 25 and FIGS. 26A-26F. The launch point geometries included flat (FIG. 26A), oval (FIG. 26B), round (FIG. 26C), square (FIG. 26D), trough (FIG. 26F), and V-shaped (FIG. 26E). Each of the launch point geometries were tested with at least two sets of dimension parameters that describe the launch point geometry, described elsewhere herein. The results of the simulated ray tracing and optical illumination transmission are shown in FIG. 25 and FIGS. 26A-26E, respectively. From FIG. the V-shaped geometries illumination uniformity performed superior to all other launch point geometries with approximately 33-34% of the injected light source power at a point before the exit tip of the speculum. With regards to power transmission, the oval (FIG. 26B) geometry performed the best.

### Example 8: In-vivo Illumination Comparison of Speculum Tips of Various Post Processing Approaches

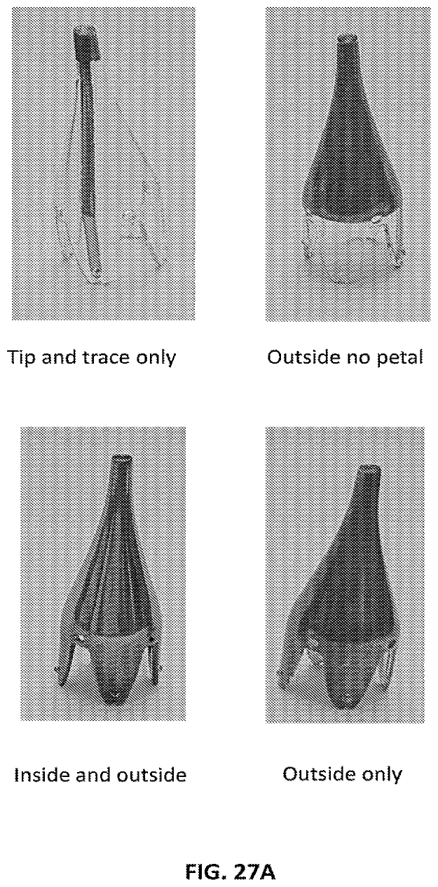

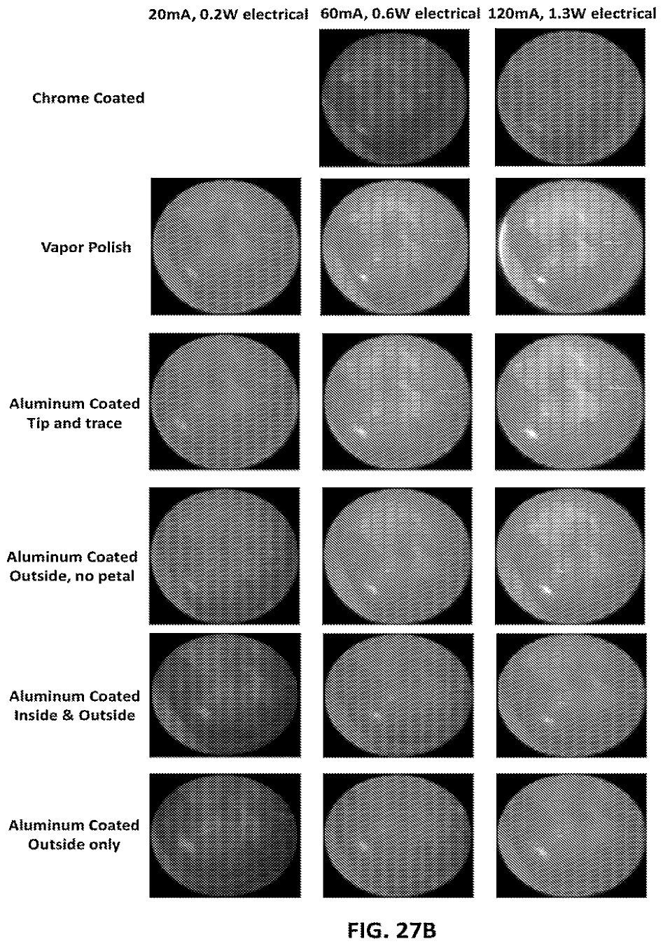

In-ear videos were taken using illuminated speculum tips where the speculum tips were chrome coated (FIG. 17B), vapor polished (FIG. 17A), or aluminum coated (FIG. 27A). For purposes of this experiment, various aluminum coated geometries, shown in FIG. 27A were analyzed, namely: coating of the speculum exit tip and lead wire trace of the XMC insert (tip and trace), coating the external surface of conical section of the speculum tip (outside no petal), coating all inside and outside surfaces of the speculum tip except the exit tip surface and light source launch points (inside and outside), and coating the entire external surface of the speculum tip (outside only).

A Hawkeye Pro Super Slim borescope and iPhone 12 camera were used to capture videos of an illuminated tympanic membrane of a subject as the drive current to the illumination LEDs was varied. The iPhone 12 camera exposure settings were locked to the following set points: ISO 100, EV 0, Color Temp 4000K, Shutter speed 1/60 sec, manual focus 50. The Hawkeye focus was adjusted to give the sharpest image while the live in-ear video is being recorded. Representative images from each of the experimental conditions (i.e., varying current light source supply values) are shown in FIG. 27B for the various speculum tips

From the images shown in FIG. 27B, the brightness and clarity of the image of the tympanic membrane at low current and electrical power is highest for the vapor polished, and tip and trace aluminum coated speculum tips.

### Example 9: Comparing Illumination Pattern and Power of a Petal and a Commercial Speculum Tip

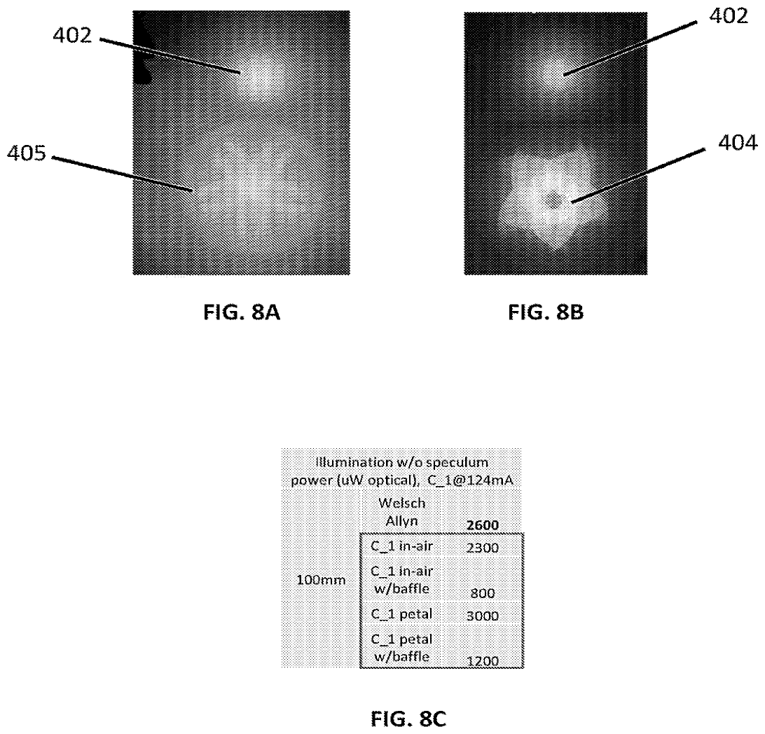

FIGS. 8A-8C show a comparison of spatial illumination patterns and transmitted power of a petal speculum tip (404), conical speculum tip without a distal petal structure (405), described elsewhere herein, and a commercial (Welsch Allyn) speculum tip (402). An LED light source was utilized as the illumination light source for the purpose of the analysis. For the petal speculum tip, the LED light source was coupled to the petal speculum tip by placing the LED light source in contact or in near contact with the contoured petal launch point, described elsewhere herein. For the conical speculum tip without a distal petal structure, the LED light source was coupled into the conical speculum tip by a parabolic mirror configured to collimate the LED light source prior to entering the conical speculum tip. Spatial illumination patterns for each speculum tip were measured at a distance of 100 mm from the speculum tip exit surface shown in FIGS. 8A and 8B, respectively. From the images in FIGS. 8A and 8B, the spatial illumination pattern for the petal speculum tip (404), and the conical speculum tip without the distal petal structure (405) appear to be larger in area of illumination as compared to the Welsch Allyn illumination pattern 402. Additionally, the petal speculum tip illumination 404 seems to produce an illumination pattern with greater spatial illumination uniformity as compared to the other speculum tips (402, 405). Such an improvement in illumination area and uniformity may provide the advantage of improving the alignment of the ultrasound transducer, described elsewhere herein, and subsequent acquisition of data.

Turning to FIG. 8C, a measurement of optical power at 100 mm from the speculum tip may be seen. The optical power data shown in FIG. 8C highlights and underscores the superior performance and light coupling efficiency of the “petal” speculum tip with the light conducting elements combined with a conical section as compared to the conical speculum tip without the distal petal structure and Welsch Allyn speculum tip.

FIGS. 12A-12B compare the diffuse illumination pattern of the petal (504) and commercial Welsch Allyn speculum tips (502) at the distal exit tip (FIG. 12A) and at 25 mm from the distal exit tip (FIG. 12B). From the images shown, the petal speculum tip 504 has a larger spatially uniform and diffuse pattern when compared to the Welsch Allyn 502 speculum tip. Additionally, seen in FIG. 12B at 25 mm from the distal tip of the Welsch Allyn 502, a dark spot of artifact can be seen at the center of the illumination 502 that would otherwise limit the uniformity of the illumination and overall optical power transmitted to the surface imaged.

FIG. 12C shows a macro perspective image taken of an illuminated C0 (508), described elsewhere herein, and a petal shaped speculum tip (504). From the image the brightness of the petal shaped speculum tip (504) may be seen to be five-times brighter than the illumination of the C0 speculum tip (508) under the same light source drive current settings.

### Example 10: Comparing Illumination Pattern of Coated Speculum Tips

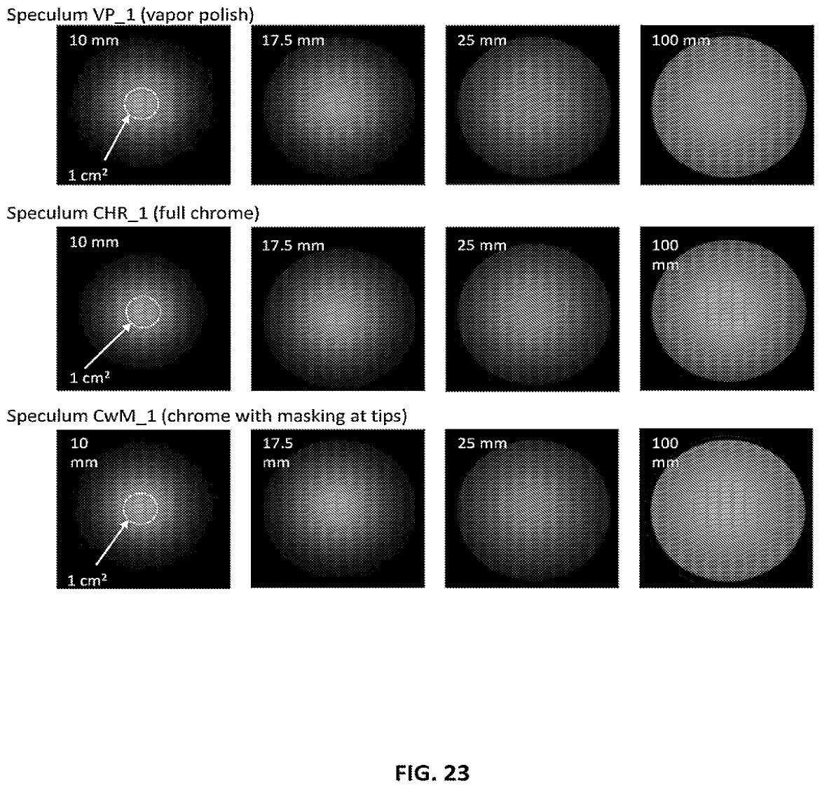

FIG. 23 shows images of various processed speculum tips at various distances (e.g., 10 mm, 17.5 mm, 25 mm, or 100 mm) from the speculum tip distal exit tip, described elsewhere herein, e.g., Example 6. For purposes of interpreting the data of FIG. 23, VP_1 corresponds to a vapor polished speculum tip, intended to imitate, or model the performance of a speculum tip from a tooled manufactured speculum tip. CHR_1 indicates a speculum tip with chrome coating on all surfaces except the one or more launch points and the exit tip distal surface. CwM_1 corresponds to a speculum tip with a chrome coating applied to all interior and exterior surface of the speculum tip without coating the multiple light source launch points, distal exit tip of the speculum, and a mask region on the side walls near the distal exit tip of the speculum. From FIG. 23 the vapor polished speculum tip appears to provide the most uniform illumination pattern when compared to the chrome coated, and partially chrome coated speculum tips.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

Although the above steps show each of the methods in accordance with embodiments, a person of ordinary skill in the art will recognize many variations based on the teaching described herein. The steps may be completed in a different order. Steps may be added or omitted. Some of the steps may comprise sub-steps. Many of the steps may be repeated as often as beneficial.

One or more of the steps of each of the methods may be performed with circuitry as described herein, for example, one or more of the processor or logic circuitry such as programmable array logic for a field programmable gate array. The circuitry may be programmed to provide one or more of the steps of each of the methods, and the program may comprise program instructions stored on a computer readable memory or programmed steps of the logic circuitry such as the programmable array logic or the field programmable gate array, for example.

What is claimed is:

1. A speculum operable to be disposed within an ear of a subject, the speculum comprising:
a housing comprising a light conducting element, wherein a transmitted optical illumination is conducted by total internal reflection via the light conducting element, wherein the housing has a lumen therewithin, and wherein the housing is configured to allow a reflected optical illumination to propagate through the lumen; and
one or more coupling portions which couple the transmitted optical illumination from a light source to the light conducting element, wherein the one or more coupling portions are shaped as a conic section.

2. The device of claim 1, further comprising an insert, wherein the insert is configured to be mechanically coupled to the housing.

3. The device of claim 2, wherein the insert comprises a lens, an ultrasound transducer, one or more electrical leads electrically coupled to the ultrasound transducer, one or more wires electrically coupled to the one or more electrical leads and the ultrasound transducer, or any combination thereof.

4. The device of claim 3, wherein the ultrasound transducer comprises a capacitive micromachined ultrasonic transducer.

5. The device of any one of claims 1-3, wherein the light conducting element comprises an ellipsoid shape.

6. The device of any one of claims 1-5, wherein the light conducting element is configured to be a parabolic mirror when light rays of the light source interact with the light conducting element.

7. The device of any one of claims 1-6, wherein the light conducting element comprises a launch point.

8. The device of any one of claims 1-6, wherein the light conducting element comprises one or more launch points.

9. The device as in claim 7 or 8, wherein the launch points comprise a geometry, wherein the geometry comprises: flat, round, oval, trough, square, or V-shaped.

10. The device of claim 1, wherein the housing comprises a proximal sealing member, a distal sealing member, or any combination thereof.

11. The device of any one of claims 1-10, wherein the proximal sealing member and the distal sealing member comprise an elastomeric material configured to seal the housing within the ear of the subject.

12. The device of claim 3, wherein the ultrasound transducer is electrically coupled to the one or more electric leads of the insert by the one or more wires.

13. The device of any one of claims 2-12, wherein the insert comprises a spacer structure configured to space the insert from the internal surface of the lumen of the housing.

14. The device of any one of claims 2-13, wherein the insert comprises a structure configured to releasably couple to the housing when inserted into the housing.

15. The device of claim 14, wherein the structure comprises a groove, hole, or hook configured to snap to a structure of the housing.

16. The device of any one of claims 1-15, wherein the housing is partially or wholly vapor polished, aluminum coated, chrome coated, or any combination thereof.

17. The device of any one of claims 1-16, wherein the insert comprises an electrical coupling interface comprising an electro-mechanical structure configured to releasably couple with and be in electrically communication with a receptacle.

18. The device of claim 17, wherein the electro-mechanical structure comprises one or more electrical pads adjacent a surface of one or more mechanical coupling interfaces.

19. The device of claim 18, wherein the one or more mechanical coupling interfaces comprises a hook configured to couple with a clasp receptable.

20. An otoscope, the otoscope comprising:
a speculum and having a lumen therewithin and comprising a light conducting element, wherein a transmitted optical illumination is conducted by total internal reflection by the light conducting element, and wherein a reflected optical illumination is propagated through the lumen of the speculum; and
one or more coupling portions which couple the transmitted optical illumination from a light source to the light conducting element, wherein the one or more coupling portions are shaped as a conic section.

21. The device of claim 20, further comprising an insert, wherein the insert is configured to mechanically couple to the speculum.

22. The device of claim 21, wherein the insert comprises a lens, an ultrasound transducer, one or more electrical leads electrically coupled to the ultrasound transducer, one or more wires electrically coupled to the one or more electrical leads and the ultrasound transducer, or any combination thereof.

23. The device of claim 22, wherein the ultrasound transducer comprises a capacitive micromachined ultrasonic transducer.

24. The device of any one of claims 20-23, wherein the light conducting element comprises an ellipsoid shape.

25. The device of any one of claims 20-24, wherein the light conducting element is configured to be a parabolic mirror when light rays of the light source interact with the light conducting element.

26. The device of any one of claims 20-25, wherein the light conducting element comprises a launch point.

27. The device of any one of claims 20-25, wherein the light conducting element comprises at least two launch points.

28. The device as in claim 26 or 27, wherein the launch point comprises a geometry, wherein the geometry comprises: flat, round, oval, trough, square, or V-shaped.

29. The device of any one of claims 20-28, wherein the speculum comprises a proximal sealing member, a distal sealing member, or any combination thereof.

30. The device of claim 29, wherein the proximal sealing member and the distal sealing member comprise an elastomeric material configured to seal the housing within the ear of the subject.

31. The device of any one of claims 22-30, wherein the ultrasound transducer is electrically coupled to the one or more electric leads of the insert by the one or more wires.

32. The device of any one of claims 22-31, wherein the insert comprises a spacer structure configured to space the insert from the internal surface of the lumen of the speculum.

33. The device of any one of claims 22-32, wherein the insert comprises a structure configured to releasably couple to the speculum when inserted into the speculum.

34. The device of claim 33, wherein the structure comprises a groove, hole, or hook configured to snap to a structure of the speculum.

35. The device of any one of claims 20-34, wherein the speculum is partially or wholly vapor polished, aluminum coated, chrome coated, or any combination thereof.

36. The device of any one of claims 22-35, wherein the insert comprises an electrical coupling interface comprising an electro-mechanical structure configured to releasably couple with and be in electrically communication with a receptacle.

37. The device of claim 36, wherein the electro-mechanical structure comprises one or more electrical pads adjacent a surface of one or more mechanical coupling interface.

38. The device of claim 37, wherein the one or more mechanical coupling interface comprises a hook configured to couple with a clasp receptable.

39. A method of using an otoscope, the method comprising:
directing optical illumination of a light source to one or more coupling portions, wherein the one or more coupling portions are shaped as a conic section;
collimating the optical illumination using the one or more coupling portions;
directing the optical illumination from the one or more coupling portions to a light conducting element, wherein the optical illumination propagates through the light conducting element by total internal reflection; and
collecting reflected optical illumination from a target within a lumen of a housing, wherein the housing comprises a portion of a speculum of an otoscope.

40. The method of claim 39, further comprising directing pneumatic excitation toward a target.

41. The method of claim 40, further comprising directing ultrasound or illumination toward the target.

42. The method of claim 40, further comprising measuring a response of the target to the pneumatic excitation in a reflected ultrasound signal.

43. The method of any one of claims 39-42, further comprising determining a state or condition of a subject based on the reflected optical illumination and the response.
